SEQUENCE LISTING (36 sequences)

SEQ ID NO: 1 - protein, 524 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHL

SEQ ID NO: 2 - protein, 133 aa, Homo sapiens [sequence omitted]

SEQ ID NO: 3 - protein, 6 aa, Artificial Sequence
  Description of Artificial Sequence Synthetic peptide
GGYLRQ

SEQ ID NO: 4 - protein, 6 aa, Artificial Sequence
  Description of Artificial Sequence Synthetic peptide
GGYLKQ

SEQ ID NO: 5 - protein, 6 aa, Artificial Sequence
  Description of Artificial Sequence Synthetic peptide
GGYRHQ

SEQ ID NO: 6 - protein, 11 aa, Artificial Sequence
  Description of Artificial Sequence Synthetic peptide
DICLPRWGCLW

SEQ ID NO: 7 - protein, 6 aa, Artificial Sequence
  Description of Artificial Sequence Synthetic peptide
GGYFKQ

SEQ ID NO: 8 - protein, 6 aa, Artificial Sequence
  Description of Artificial Sequence Synthetic peptide
GGYLPQ

SEQ ID NO: 9 - protein, 6 aa, Artificial Sequence
  Description of Artificial Sequence Synthetic peptide
GGYMPQ

SEQ ID NO: 10 - protein, 7 aa, Artificial Sequence
  Description of Artificial Sequence Synthetic peptide
GGYDKPH

SEQ ID NO: 11 - protein, 4 aa, Artificial Sequence
  Description of Artificial Sequence Synthetic peptide
YLRQ

SEQ ID NO: 12 - protein, 4 aa, Artificial Sequence
  Description of Artificial Sequence Synthetic peptide
YLKQ

SEQ ID NO: 13 - protein, 4 aa, Artificial Sequence
  Description of Artificial Sequence Synthetic peptide
YRHQ

SEQ ID NO: 14 - protein, 4 aa, Artificial Sequence
  Description of Artificial Sequence Synthetic peptide
YLRQ

SEQ ID NO: 15 - protein, 4 aa, Artificial Sequence
  Description of Artificial Sequence Synthetic peptide
YFKQ

SEQ ID NO: 16 - protein, 4 aa, Artificial Sequence
  Description of Artificial Sequence Synthetic peptide
YLPQ

SEQ ID NO: 17 - protein, 4 aa, Artificial Sequence
  Description of Artificial Sequence Synthetic peptide
YMPQ

SEQ ID NO: 18 - protein, 5 aa, Artificial Sequence
  Description of Artificial Sequence Synthetic peptide
YDKPH

SEQ ID NO: 19 - protein, 4 aa, Artificial Sequence
  Description of Artificial Sequence Synthetic peptide
  MOD_RES (2)..(3): Any natural amino acid
  See specification as filed for detailed description of substitutions and preferred embodiments
YXXL

SEQ ID NO: 20 - protein, 4 aa, Artificial Sequence
  Description of Artificial Sequence Synthetic peptide
YLSL

SEQ ID NO: 21 - protein, 4 aa, Artificial Sequence
  Description of Artificial Sequence Synthetic peptide
  MOD_RES (2)..(3): Any amino acid
  See specification as filed for detailed description of substitutions and preferred embodiments
YXXQ

SEQ ID NO: 22 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 23 - protein, 132 aa, Artificial Sequence
  Description of Artificial Sequence Synthetic polypeptide
PTSSSTKKTQLQLSQLLVLLKAILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 24 - protein, 489 aa, Artificial Sequence
  Description of Artificial Sequence Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 1060 aa, Artificial Sequence
  Description of Artificial Sequence Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPGSGEGRGSLLTCGDVEENPGPMAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 26 - protein, 1066 aa, Artificial Sequence
  Description of Artificial Sequence Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPGSGEGRGSLLTCGDVEENPGPMAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGGYRHQ

SEQ ID NO: 27 - protein, 5 aa, Homo sapiens
YYLSL

SEQ ID NO: 28 - protein, 20 aa, Artificial Sequence
  Description of Artificial Sequence Synthetic peptide
  SITE (1)..(20): This sequence may encompass 1-10 “Gly Gly” repeating units
GGGGGGGGGGGGGGGGGGGG

SEQ ID NO: 29 - protein, 4 aa, Homo sapiens
YFTY

SEQ ID NO: 30 - protein, 4 aa, Homo sapiens
YDPY

SEQ ID NO: 31 - protein, 4 aa, Homo sapiens
YSEE

SEQ ID NO: 32 - protein, 133 aa, Artificial Sequence
  Description of Artificial Sequence Synthetic polypeptide
  MOD_RES (1)..(1): A or not present
  MOD_RES (2)..(2): P or not present
  MOD_RES (3)..(3): T, C, A, G, Q, E, N, D, R, K, P, or not present
  MOD_RES (4)..(4): S or not present
  MOD_RES (5)..(5): S or not present
  MOD_RES (6)..(6): S or not present
  MOD_RES (7)..(7): T or not present
  MOD_RES (8)..(8): K or not present
  MOD_RES (9)..(9): K or not present
  MOD_RES (13)..(13): Q, W, or not present
  MOD_RES (14)..(14): L, M, W, or not present
  MOD_RES (15)..(15): E, K, D, T, A, S, Q, H, or not present
  MOD_RES (16)..(16): H, N, Q, or not present
  MOD_RES (18)..(18): L, R, G, M, F, E, H, W, K, Q, S, V, I, Y, H, D, or T
  MOD_RES (19)..(19): L, A, V, I, or not present
  MOD_RES (20)..(20): D, T, S, M, L, or not present
  MOD_RES (22)..(22): Q, F, E, G, A, L, M, F, W, K, S, V, I, Y, H, R, N, D, T, F, or not present
  MOD_RES (23)..(23): M, A, W, H, Y, F, Q, S, V, L, T, or not present
  MOD_RES (27)..(27): G, K, S, or not present
  MOD_RES (38)..(38): R, W, or G
  MOD_RES (39)..(39): M, L, or V
  MOD_RES (42)..(42): F or K
  MOD_RES (51)..(51): T, I, or not present
  MOD_RES (55)..(55): H or Y
  MOD_RES (74)..(74): Q, N, H, or S
  MOD_RES (80)..(80): L, F, or V
  MOD_RES (81)..(81): R, I, D, Y, T, or not present
  MOD_RES (85)..(85): L or V
  MOD_RES (86)..(86): I or V
  MOD_RES (89)..(89): I or V
  MOD_RES (91)..(91): V, R, or K
  MOD_RES (92)..(92): I or F
  MOD_RES (97)..(97): K or Q
  MOD_RES (104)..(104): M or A
  MOD_RES (109)..(109): D, C, or a non-natural amino acid with an activated side chain
  MOD_RES (113)..(113): T or N
  MOD_RES (125)..(125): C, A, or S
  MOD_RES (126)..(126): Q, H, M, K, C, D, E, G, I, R, S, or T
  MOD_RES (130)..(130): S, T, or R
XXXXXXXXXTQLXXXXLXXXLXXILNXINNYKNPKLTXXLTXKFYMPKKAXELKXLQCLEEELKPLEEVLNLAXSKNFHXXPRDXXSNXNXXVLELXGSETTFXCEYAXETAXIVEFLNRWITFXXSIIXTLT

SEQ ID NO: 33 - protein, 6 aa, Artificial Sequence
  Description of Artificial Sequence Synthetic His tag
  SITE (1)..(6): This sequence may encompass 3-6 residues
HHHHHH

SEQ ID NO: 34 - protein, 6 aa, Artificial Sequence
  Description of Artificial Sequence Synthetic 6xHis tag
HHHHHH

SEQ ID NO: 35 - protein, 10 aa, Artificial Sequence
  Description of Artificial Sequence Synthetic peptide
  SITE (1)..(10): This sequence may encompass 1-10 residues
  See specification as filed for detailed description of substitutions and preferred embodiments
GGGGGGGGGG

SEQ ID NO: 36 - protein, 10 aa, Homo sapiens
YFTYDPYSEE

(19) United States
(12) Patent Application Publication
(10) Pub. No.: US 2023/0027899 A1
(43) Pub. Date: Jan. 26, 2023

(54) CD122 WITH ALTERED ICD STAT SIGNALING

(71) Applicant: Synthekine, Inc., Menlo Park, CA (US)

(72) Inventors: Paul-Joseph Penaflor Aspuria, Menlo Park, CA (US); Patrick J. Lupardus, Menlo Park, CA (US)

(21) Appl. No.: 17/783,505

(22) PCT Filed: Jan. 14, 2021
(86) PCT No.: PCT/US2021/013519

§ 371 Date: Jun. 8, 2022

Related U.S. Application Data
(60) Provisional application No. 62/961,157, filed on Jan. 14, 2020.

(51) Int. Cl. C07K 14/55; C07K 14/715; C07K 14/725; C12N 15/63

(52) U.S. Cl. CPC C07K 14/55; C07K 14/7155; C07K 14/7051; C12N 15/63

(57) ABSTRACT

The present disclosure relates to modified human CD122, wherein the modified human CD122 comprises one or more STAT3 binding motifs. In some embodiments, the modified human CD122 is a modified orthogonal human CD122, which can be selectively activated by a cognate orthogonal IL2. The modified human CD122 is able to stimulate robust and sustained STAT3 and STAT5 signaling upon binding to a cognate IL2 ligand.

### RELATED APPLICATION

This application claims priority to and the benefit of U.S. Provisional Application No. 62/961,157, filed on Jan. 14, 2020. The entire content of said provisional application is herein incorporated by reference for all purposes.

### BRIEF DESCRIPTION OF THE FIGURES

FIG. 1 provides a diagrammatic illustration of an orthogonal CD122 (IL2Rb) polypeptide in association with a cell membrane (left) and one configuration of a representative embodiment of the engineered receptors of the disclosure wherein the intracellular domain of the orthogonal CD122 peptide comprising the STAT3 motif YRHQ is added to the carboxy terminus of the via a glycine-glycine linker (right).

FIG. 2 provides the results of a FACS study confirming the efficient transduction and expression of the indicated orthogonal CD122 (hoRb, center panel) and the orthogonal CD122 comprising an additional STAT3 motif (right panel) are efficiently introduced and expressed in the 3F8 cells.

FIG. 3 provides the results of a FACS study evaluating the relative percentage of 3F8 T cell clones transduced with the indicated orthogonal CD122 (hoRb) and the orthogonal CD122 comprising an additional STAT3 motif have elevated CD25 and CD122 expression in response to the orthogonal ligand.

FIG. 4 provides the results of an evaluation of phosphoSTAT3 (pSTAT3) levels in CD4+ T cells (upper left), CD8+ T cells (upper right) engineered to express orthogonal hoRb receptor constructs comprising the IL2 wtIL2Rb ICD in response to wtIL2 (black squares), the wt IL2R ICD in response to the administration of an orthogonal ligand STK-009 (grey circles) and the IL2 wtIL2Rb ICD with addition of a STAT3 motif to the administration of an orthogonal ligand STK-009; and phosphoSTAT5 (pSTAT5) levels in CD4+ T cells (lower left), CD8+ T cells (lower right) engineered to express orthogonal hoRb receptor constructs comprising the IL2 wtIL2Rb ICD in response to wtIL2 (black squares), the wt IL2R ICD in response to the administration of an orthogonal ligand STK-009 (grey circles) and the IL2 wtIL2Rb ICD with addition of a STAT3 motif to the administration of an orthogonal ligand STK-009.

FIG. 5 provides the results of an evaluation of pERK signaling levels in CD4+ T cells (upper left), CD8+ T cells (upper right) engineered to express orthogonal hoRb receptor constructs comprising the IL2 wtIL2Rb ICD in response to wtIL2 (black squares), the wt IL2R ICD in response to the administration of an orthogonal ligand STK-009 (grey circles) and the IL2 wtIL2Rb ICD with addition of a STAT3 motif to the administration of an orthogonal ligand STK-009; and pS6K (pSTAT5) levels in CD4+ T cells (lower left), CD8+ T cells (lower right) engineered to express orthogonal hoRb receptor constructs comprising the IL2 wtIL2Rb ICD in response to wtIL2 (black squares), the wt IL2R ICD in response to the administration of an orthogonal ligand STK-009 (grey circles) and the IL2 wtIL2Rb ICD with addition of a STAT3 motif to the administration of an orthogonal ligand STK-009.

FIG. 6 provides a graphical illustration of the results of an evaluation of the cytotoxicity evaluation of CD19 CAR T cell constructs comprising orthogonal CD122 (hoRb) receptors with an additional STAT3 signaling motif as compared to CD19 CART cell constructs comprising orthogonal CD122 (hoRb) with the wild type CD122 intracellular domain at various effector:target (E:T; CART:Raji tumor cell) ratios from 10:1 to 0.1:1 demonstrating the improved cytotoxicity of a CD19 CAR T cell construct expressing an hoRb with an intracellular domain (ICD) expressing a STAT3 motif against Raji tumor cells relative to aCD19 CART cell comprising orthogonal CD122 (hoRb) with the wild type CD122 intracellular domain.

### BACKGROUND OF THE INVENTION

The controlled manipulation of the differentiation, development and proliferation of cells, particularly engineered immune cells, is of significant clinical interest. T cells have been engineered for use in therapeutic applications such as the recognition and killing of cancer cells, intracellular pathogens, and cells involved in autoimmunity. The use of engineered cell therapies in the treatment of cancer is facilitated by the selective activation and expansion of engineered T cells that provide specific functions and are directed to selectively attack cancer cells. In some examples of adoptive immunotherapy, T cells are isolated from the blood of a subject, processed ex vivo, and re-infused into the subject. Compositions and methods that enable selective activation of a targeted engineered cell population are therefore desirable.

A challenge with the manufacture of cell therapy products is that such “living drugs” require close control of their environment to preserve viability and functionality. In practice, isolated cells, whether derived from a patient (autologous) or from a single donor source (allogeneic), begin to lose function rapidly following removal from the subject or the controlled culture conditions. Successful maintenance of the health and function of isolated cells while outside the subject or controlled culture conditions enables the isolated cells to return to functionality for reinsertion into the cell product manufacturing workflow or into patients.

Additionally, a challenge with the clinical application of engineered T cell therapies is to selectively stimulate these engineered cells to maximize their therapeutic effectiveness. Typical means to provide for the continued maintenance of activated engineered T cell products is the systemic administration of cytokines such as IL2. However, the systemic administration of IL2 is associated with non-specific stimulatory effects beyond the population of engineered cells and is associated, particularly in high doses, and is associated with significant toxicity in human subjects. Furthermore, IL2 has a short lifespan in vivo which requires that the IL2 be dosed frequently to maintain the engineered T cells in an activated state. Although engineered cells from an initial administration of an initial population may be detectable for months or even years following the administration of the engineered cell product, a significant fraction of these engineered cells lapse into a quiescent state that requires reactivation for them to exhibit significant therapeutic effect. Consequently, a challenge in cell-based therapies is to confer a desired regulatable behavior into the transferred cells that is protected from endogenous signaling pathways, that does not affect non-targeted endogenous cells, and that can be controlled selectively following administration of the engineered cell population to a subject.

CD122 is a component of the intermediate and high affinity IL2 receptor complexes. CD122 contains a native STAT5 recognition motif. Upon binding of the IL2, the receptor activates JAK (kinase), which phorphorylates certain tyrosines in the intracellular domain of the CD122. The phorphorylated CD122 recruits and phosphorylates STAT5 (STAT5A and/or STAT5B). The phosphorylated STAT5 are then dimerized and tranlocated into nucleus to activate transcription of target genes that play important functions in a variety of pathways, from innate and acquired immunity to cell proliferation, differentiation and survival. Basham et al., Nucleic Acids Res. 2008 June; 36(11): 3802-3818.

Recently, cell therapies involving T cells that have been transformed with recombinant CD122 has been developed. Sockolosky, et al. (Science (2018) 359: 1037-1042) and Garcia, et al. (United States Patent Application Publication US2018/0228841A1 published Aug. 16, 2018) describe an orthogonal IL2/CD122 ligand/receptor system to facilitate selective stimulation of cells engineered to express the orthogonal CD122. The contact of engineered T cells that express the orthogonal CD122 with a corresponding orthogonal ligand for such orthogonal CD122 (“orthologonal IL2”) enables specific activation of such engineered T cells. In particular this orthogonal IL2 receptor ligand complex provides for selective expansion of cells engineered to express the orthogonal receptor in a mixed population of cells, in particular a mixed population of T cells.

Orthogonal IL2 with diminished affinity for the non-engineered intermediate affinity (CD122/CD132) IL2 receptor complex or high-affinity (CD25/CD122/CD132) IL2 receptor complex are also useful to selectively target the activity of orthologonal IL2 towards cells which exhibit high expression of CD25, e.g. in the treatment of autoimmune disease. orthologonal IL2 with significantly diminished affinity for the native wild-type CD122 extracellular domain (ECD) but retain binding to the ECD of CD25 may also be used as competitive antagonists of wild-type IL2 by interfering with the high-affinity IL2 receptor complex formation and consequently may be employed in the treatment of autoimmune diseases or graft-versus-host (GVH) disease.

### BRIEF SUMMARY OF THE INVENTION

The present disclosure is directed to a modified human CD122, which retains a STAT5 motif as in the native human CD122 but has been engineered to comprise one or more STAT3 binding motifs. In some embodiments, the presence of STAT3 binding motifs renders the modified CD122 more stable, which can promote sustained and robust STAT3 and STAT5 signaling upon binding to a cognate IL2 ligand. In some embodiments, the modified human CD122 is a modified orthologonal human CD122, which can be selectively activated by a cognate orthologonal IL2. Thus the disclosure also provides compositions and methods for selective activation of immune cells that have been engineered to express a modified orthologonal human CD122 to promote stable and sustained IL2-mediated signaling. The methods described herein can be used to effectively treat patients in need of IL2 therapy without causing significant adverse effect that is associated with standard IL2 therapy.

This application incorporates by reference the disclosures of WO 2019/104092 and US 2018-0228842 A1) in their entireties.

In some embodiments, this disclosure provides a polynucleotide encoding a modified human CD122, wherein the modified human CD122 comprises one or more STAT3 binding motifs. In some embodiments, the modified human CD122 comprises a orthogonal human CD122 or native human CD122 fused to one or more STAT3 binding motifs. In some embodiments, the orthogonal human CD122 is modified at one or more residues selected from R41, R42, Q70, K71, T73, T74, V75, 5132, H133, Y134, F135, E136, and Q214 relative to native human CD122. In some embodiments, the orthogonal human CD122 is modified at H133 and Y134 relative to native human CD122. In some embodiments, the human CD122 is linked to two or three STAT3 binding motifs.

In some embodiments, the modified human CD122 comprises a sequence that is at least 90% identical to SEQ ID NO: 1, wherein the modified human CD122 binds to a native IL2 polypeptide or an orthogonal IL2 polypeptide. In some embodiments, the one or more STAT3 binding motifs comprise a sequence of YX1X2Q, wherein X1 and X2 are any amino acid acids. In some embodiments, X1 is selected from the group consisting of L, R, F, M, and X2 is selected from the group consisting of R, K, H, and P. In some embodiments, the STAT3 recognition motif is selected from the group consisting of YLRQ (SEQ ID NO:11), YLKQ (SEQ ID NO: 12), YRHQ (SEQ ID NO: 13), YLRQ (SEQ ID NO: 14), YFKQ (SEQ ID NO: 15), YLPQ (SEQ ID NO: 16), YMPQ (SEQ ID NO: 17), and YDKPH (SEQ ID NO: 18).

In some embodiments, the one or more STAT3 binding motifs are fused to a C-terminus of the intracellular domain of the native human CD122 or the orthogonal human CD122, optionally through a linker. In some embodiments, at least one of the STAT3 binding motifs is located between position 355 and position 364 corresponding to native human CD122, and wherein the at least one of the STAT3 recognition motif replaces an amino acid sequence of YFTY, YDPY, or YSEE in native human CD122. In some embodiments, the linker comprises a dinucleotide (GG)n, wherein n is 1 to 10.

In some embodiments, the modified human CD122 is further modified at one or more residues selected from R41, R42, Q70, K71, T73, T74, V75, 5132, H133, Y134, F135, E136, and Q214 relative to native human CD122.

In some embodiments, the modified CD122 comprises an amino acid sequence comprising the linker and at least one of the STAT3 binding motifs, wherein the amino acid sequence is selected from the group consisting of: GGYLRQ (SEQ ID NO:3), GGYLKQ (SEQ ID NO: 4), GGYRHQ (SEQ ID NO: 5), GGYLRQ (SEQ ID NO: 6), GGYFKQ (SEQ ID NO: 7), GGYLPQ (SEQ ID NO: 8), GGYMPQ (SEQ ID NO: 9), and GGYDKPH (SEQ ID NO: 10).

Also provided herein is an expression vector comprising the polynucleotide of any one of the embodiments above.

Also provided herein is an expression vector comprising the polynucleotide of any one of the embodiments above. In some embodiments, the cell further expresses a chimeric antigen receptor (CAR), and wherein the cell is a human immune cell. In some embodiments, the CAR is selected from the group consisting of a CD19 CAR and a BCMA CAR.

Also provided herein is a kit for selective activation of a receptor in a cell, the kit comprising: (a) a cell expressing a modified human CD122 encoded by the polynucleotide as disclosed herein, and (b) a human IL2 polypeptide. In some embodiments, the modified human CD122 comprises a native human CD122 encoded by SEQ ID NO: 1, and wherein the human IL2 polypeptide is a native human IL2 polypeptide encoded by SEQ ID NO: 2. In some embodiments, the modified human CD122 comprises an orthogonal human CD122, wherein the human IL2 polypeptide is an orthogonal human IL2 polypeptide, and wherein the orthogonal human IL2 polypeptide binds preferentially to the modified human CD122 compared to a native human CD122.

In some embodiments, the orthogonal human IL2 polypeptide comprises at least one amino acid substitution with an amino acid other than that of the native human IL2 polypeptide at a position corresponding to native human CD122 residue TM, R81, or comprises an alanine at a position corresponding to native human CD122 M23, and comprises amino acid substitutions at each of a position corresponding to native human CD122 E15, H16, L19, and D20.

In some embodiments, the orthogonal human IL2 polypeptide comprises one or more amino acid substitutions corresponding to native human IL2 positions selected from: [E15D, E15T, E15A, E15S], [H16N, H16Q], [L19V, L19I, L19A], [D20L, D20M], [Q22S, Q22T, Q22E, Q22K, Q22E], [M23A, M23W, M23H, M23Y, M23F, M23Q, M23Y], [G27K, G27S], [R81D, R81Y], [N88E, N88Q], [T51I].

In some embodiments, the modified human CD122 is expressed by a mammalian cell. In some embodiments, the mammalian cell is an immune cell. In some embodiments, the immune cell is a T cell. In some embodiments, the T cell is a chimeric antigen receptor (CAR)-T cell.

Also provided is a method of stimulating an immune cell expressing a modified human CD122 comprising one or more STAT3 binding motifs, the method comprising contacting the immune cell with a human IL2 polypeptide. In some embodiments, the stimulating occurs ex vivo. In some embodiments, wherein the stimulating occurs in vivo. In some embodiments, the modified human CD122 comprises a orthogonal human CD122 or native human CD122 fused to one or more STAT3 binding motifs. In some embodiments, at least one of the STAT3 binding motifs is located between position 381 and position 390 corresponding to native human CD122, and wherein the at least one of the STAT3 binding motifs replaces an amino acid sequence of YFTY, YDPY, or YSEE in the native human CD122. In some embodiments, the method comprises introducing to an individual an immune cell expressing a modified human CD122 comprising one or more STAT3 binding motifs, and administering a human IL2 polypeptide to the individual, thereby activating immune response in the individual. In some embodiments, the modified human CD122 comprises an orthogonal human CD122, and wherein the human IL2 polypeptide is an orthogonal human IL2 polypeptide, and wherein the orthogonal human IL2 polypeptide binds preferentially to and activates the modified human CD122 than to a native human CD122. In some embodiments, the one or more STAT3 binding motifs comprise a sequence of YX1X2Q, wherein X1 and X2 are any amino acid acids. In some embodiments, X1 is selected from the group consisting of L, R, F, and M, and X2 is selected from the group consisting of R, K, H, and P. In some embodiments, the orthogonal human CD122 comprises a sequence selected from the group consisting of GGYLRQ (SEQ ID NO:2), GGYLKQ (SEQ ID NO: 3), GGYRHQ (SEQ ID NO: 4), GGYLRQ (SEQ ID NO: 5), GGYFKQ (SEQ ID NO: 6), GGYLPQ (SEQ ID NO: 7), GGYMPQ (SEQ ID NO: 8), and GGYDKPH (SEQ ID NO: 9). In some embodiments, the immune cell is a T cell. In some embodiments, the T cell is a CAR-T cell. In some embodiments, the immune cell is CD8+ T cells, and wherein the individual has cancer. In some embodiments, the immune cell is Treg cells, and wherein the individual has an autoimmune disease. In some embodiments, the individual has a viral, bacterial or fungal infection.

### DETAILED DESCRIPTION OF THE INVENTION

### Definitions

In order for the present disclosure to be more readily understood, certain terms and phrases are defined below as well as throughout the specification. The definitions provided herein are non-limiting and should be read in view of the knowledge of one of skill in the art would know.

Before the present methods and compositions are described, it is to be understood that this invention is not limited to particular method or composition described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless indicated otherwise, parts are parts by weight, molecular weight is weight average molecular weight, temperature is in degrees Celsius (° C.), and pressure is at or near atmospheric. Standard abbreviations are used, including the following: bp=base pair(s); kb=kilobase(s); pl=picoliter(s); s or sec=second(s); min=minute(s); h or hr=hour(s); aa=amino acid(s); kb=kilobase(s); nt=nucleotide(s); pg=picogram; ng=nanogram; μg=microgram; mg=milligram; g=gram; kg=kilogram; dl or dL=deciliter; μl or μL=microliter; ml or mL=milliliter; 1 or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=weekly; QM=monthly; HPLC=high performance liquid chromatography; BW=body weight; U=unit; ns=not statistically significant; PBS=phosphate-buffered saline; PCR=polymerase chain reaction; NHS=N-hydroxysuccinimide; HSA=human serum albumin; MSA=mouse serum albumin; DMEM=Dulbeco's Modification of Eagle's Medium; GC=genome copy; EDTA=ethylenediaminetetraacetic acid.

It will be appreciated that throughout this disclosure reference is made to amino acids according to the single letter or three letter codes. For the reader's convenience, the single and three letter amino acid codes are provided in Table 1 below:

TABLE 1
Amino Acid Abbreviations

| | | |
|---|---|---|
| G | Glycine | Gly |
| P | Proline | Pro |
| A | Alanine | Ala |
| V | Valine | Val |
| L | Leucine | Leu |
| I | Isoleucine | Ile |
| M | Methionine | Met |
| C | Cysteine | Cys |
| F | Phenylalanine | Phe |
| Y | Tyrosine | Tyr |
| W | Tryptophan | Trp |
| H | Histidine | His |
| K | Lysine | Lys |
| R | Arginine | Arg |
| Q | Glutamine | Gln |
| N | Asparagine | Asn |
| E | Glutamic Acid | Glu |
| D | Aspartic Acid | Asp |
| S | Serine | Ser |
| T | Threonine | Thr |

Standard methods in molecular biology are described in the scientific literature (see, e.g., Sambrook and Russell (2001) Molecular Cloning, 3rd ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)). The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

Unless otherwise indicated, the following terms are intended to have the meaning set forth below. Other terms are defined elsewhere throughout the specification.

As used herein the term “activate” is used in reference to a receptor or receptor complex to reflect the biological effect of the binding of an agonist ligand to the receptor. For example, it is said that the binding of IL2 agonist to the IL2 receptor “activates” the signaling of the receptor to produce one or more intracellular biological effects (e.g., phosphorylation of STAT5).

As used herein, the term “activity” is used with respect to a molecule to describe a property of the molecule with respect to a test system or biological function such as the degree of binding of the molecule to another molecule. Examples of such biological functions include but are not limited to catalytic activity of a biological agent, the ability to stimulate intracellular signaling, gene expression, cell proliferation, the ability to modulate immunological activity such as inflammatory response. “Activity” is typically expressed as a biological activity per unit of administered agent such as [catalytic activity]/[mg protein], [immunological activity]/[mg protein], international units (IU) of activity, [STAT5 or STAT3 phosphorylation]/[mg protein], [T-cell proliferation]/[mg protein], plaque forming units (pfu), etc. The term “proliferative activity” encompasses an activity that promotes cell division including dysregulated cell division as that observed in neoplastic diseases, inflammatory diseases, fibrosis, dysplasia, cell transformation, metastasis, and angiogenesis.

The terms “administration” and “administer” are used interchangeably herein to refer the act of contacting a subject, including contacting a cell, tissue, organ, or biological fluid in vitro, in vivo or ex vivo of the subject, with an agent (e.g. an orthologonal IL2, a CAR-T cell, a chemotherapeutic agent, an antibody, or modulator or a pharmaceutical formulation comprising one or more of the foregoing). Administration of an agent may be achieved through any of a variety of art recognized methods including but not limited to the topical, intravascular injection (including intravenous or intraarterial infusion), intradermal injection, subcutaneous injection, intramuscular injection, intraperitoneal injection, intracranial injection, intratumoral injection, transdermal, transmucosal, iontophoretic delivery, intralymphatic injection, intragastric infusion, intraprostatic injection, intravesical infusion (e.g., bladder), respiratory inhalers, intraocular injection, intraabdominal injection, intralesional injection, intraovarian injection, intracerebral infusion or injection, intracerebroventricular injection (ICVI), and the like. The term “administration” includes contact of an agent to the cell, tissue or organ as well as the contact of an agent to a fluid, where the fluid is in contact with the cell.

As used herein the term “affinity” refers to the degree of specific binding of a first molecule (e.g. a ligand) to a second molecule (e.g. a receptor) and is measured by the binding kinetics expressed as Kd, a ratio of the dissociation constant between the molecule and the its target (Koff) and the association constant between the molecule and its target (Kon).

As used herein, the term “biological sample” or “sample” refers to a sample obtained or derived from a subject. By way of example, a biological sample comprises a material selected from the group consisting of body fluids, blood, whole blood, plasma, serum, mucus secretions, saliva, cerebrospinal fluid (CSF), bronchoalveolar lavage fluid (BALF), fluids of the eye (e.g., vitreous fluid, aqueous humor), lymph fluid, lymph node tissue, spleen tissue, bone marrow, and an immunoglobulin enriched fraction derived from one or more of these tissues. In some embodiments, the sample is obtained from a subject who has been exposed to a therapeutic treatment regimen including a pharmaceutical formulation of an orthologonal IL2, such as repeatedly exposed to the same drug. In other embodiments, the sample is obtained from a subject who has not recently been exposed to the orthologonal IL2 or obtained from the subject prior to the planned administration of the orthologonal IL2.

As used herein, the terms “chimeric antigen receptor” and “CAR” are used interchangeably to refer to a chimeric polypeptide comprising multiple functional domains arranged from amino to carboxy terminus in the sequence: (a) an antigen binding domain (ABD), (b) a transmembrane domain (TD); and (c) one or more cytoplasmic signaling domains (CSDs) wherein the foregoing domains may optionally be linked by one or more spacer domains. The CAR may also further comprise a signal peptide sequence which is conventionally removed during post-translational processing and presentation of the CAR on the cell surface of a cell transformed with an expression vector comprising a nucleic acid sequence encoding the CAR. CARs useful in the practice of the present methods can be prepared in accordance with principles well known in the art. See e.g., Eshhaar et al. U.S. Pat. No. 7,741,465 B1, issued Jun. 22, 2010; Sadelain, et al (2013) Cancer Discovery 3(4):388-398; Jensen and Riddell (2015) Current Opinions in Immunology 33:9-15; Gross, et al. (1989) PNAS (USA) 86(24):10024-10028; Curran, et al. (2012) J Gene Med 14(6):405-15. Examples of commercially available CAR-T cell products that may be modified to incorporate an orthogonal receptor of the present invention include axicabtagene ciloleucel (marketed as Yescarta® commercially available from Gilead Pharmaceuticals) and tisagenlecleucel (marketed as Kymriah® commercially available from Novartis).

As used herein, the terms “chimeric antigen receptor T-cell” and “CAR-T cell” are used interchangeably to refer to a T-cell that has been recombinantly modified to express a chimeric antigen receptor. As used herein, a CAR-T cell may be engineered to express an orthogonal CD122 polypeptide.

As used herein, the term “interleukin-2” or “IL2” refers to a naturally occurring IL2 polypeptide that possesses IL2 activity. In some embodiments, IL2 refers to mature wild-type human IL2. Mature wild-type human IL2 (hIL2) occurs as a 133 amino acid polypeptide (less the signal peptide, consisting of an additional 20 N-terminal amino acids), as described in Fujita, et. al., PNAS USA, 80, 7437-7441 (1983). An amino acid sequence of naturally occurring variant of mature wild-type human IL2 (hIL2) is:

(SEQ ID NO: 2)
APTSSSTKKT QLQLEHLLLD LQMILNGINN YKNPKLTRML
TFKFYMPKKA TELKHLQCLE EELKPLEEVL NLAQSKNFHL
RPRDLISNIN VIVLELKGSE TTFMCEYADE TATIVEFLNR
WITFCQSIIS TLT

As used herein, the numbering of residues is based on the IL2 sequence UniProt ID P60568 excluding the signal peptide which is the same as that of SEQ ID NO:2.

As used herein the term “native human CD122” refers to a naturally-occurring human CD122, including naturally occurring variants thereof. The amino acid sequence of one naturally occurring human CD122 variant is:

(SEQ ID NO: 1)
AVNGTSQFTC FYNSRANISC VWSQDGALQD TSCQVHAWPD
RRRWNQTCEL LPVSQASWAC NLILGAPDSQ KLTTVDIVTL
RVLCREGVRW RVMAIQDFKP FENLRLMAPI SLQVVHVETH
RCNISWEISQ ASHYFERHLE FEARTLSPGH TWEEAPLLTL
KQKQEWICLE TLTPDTQYEF QVRVKPLQGE FTTWSPWSQP
LAFRTKPAAL GKDTIPWLGH LLVGLSGAFG FIILVYLLIN
CRNTGPWLKK VLKCNTPDPS KFFSQLSSEH GGDVQKWLSS
PFPSSSFSPG GLAPEISPLE VLERDKVTQL LLQQDKVPEP
ASLSSNHSLT SCFTNQGYFF FHLPDALEIE ACQVYFTYDP
YSEEDPDEGV AGAPTGSSPQ PLQPLSGEDD AYCTFPSRDD
LLLFSPSLLG GPSPPSTAPG GSGAGEERMP PSLQERVPRD
WDPQPLGPPT PGVPDLVDFQ PPPELVLREA GEEVPDAGPR
EGVSFPWSRP PGQGEFRALN ARLPLNTDAY LSLQELQGQD PTHL

As used herein the term “human CD122” can be a native human CD122 or an orthologonal human CD122 ortholog. As used herein, the numbering of residue with regard to human CD122 is based on SEQ ID NO: 1.

As used herein, the terms “human orthogonal CD122” or “orthogonal human CD122” are used interchangeably to refers to a variant of the native CD122 polypeptide that can specifically bind to at least one orthogonal IL2. In some embodiments, the orthogonal human CD122 comprises amino acid substitutions at positions histidine 133 (H133) and tyrosine 134 (Y134) in the ECD of the hCD122 polypeptide. In some embodiments orthogonal CD-122 comprises the amino acid substitutions at position 133 from histidine to aspartic acid (H133D), glutamic acid (H133E) or lysine (H133K) and/or amino acid substitutions at position 134 to from tyrosine to phenylalanine (Y134F), glutamic acid (Y134E), or arginine (Y134R). In some embodiments, the orthogonal CD122 is a hCD122 molecule having amino acid substitutions H133D and Y134F.

As used herein, the term “modified human CD122” refers to a protein comprising a human CD122 and one or more STAT3 binding motifs. A human CD122 may be a human orthogonal CD122 or a native human CD122. In some embodiments, the modified human CD122 retains the STAT5 motif YYLSL (SEQ ID NO: 20) as in the native human CD122.

As used herein, the term “modified human orthogonal CD122” refers to one type of the modified human CD122 protein, which comprises a human orthogonal CD122 and one or more STAT3 binding motifs. In some embodiments, the modified CD122 retains the STAT5 motif YLSL (SEQ ID NO: 20).

As used herein in the term “derived from”, in the context of an amino acid sequence or polynucleotide sequence (e.g., an amino acid sequence “derived from” an IL2 polypeptide), is meant to indicate that the polypeptide or nucleic acid has a sequence that is based on that of a reference polypeptide or nucleic acid (e.g., a naturally occurring IL2 polypeptide or an IL2-encoding nucleic acid), and is not meant to be limiting as to the source or method in which the protein or nucleic acid is made. By way of example, the term “derived from” includes homologs or variants of reference amino acid or DNA sequences.

As used herein the term “extracellular domain” or its abbreviation “ECD” refers to the portion of a cell surface protein (e.g. a cell surface receptor) which is outside of the plasma membrane of a cell. The ECD may include the entire extra-cytoplasmic portion of a transmembrane protein, a cell surface or membrane associated protein, a secreted protein, a cell surface targeting protein,

The term “IL2 activity” refers to one or more the biological effects on a cell in response to contacting the cell with an effective amount of an IL2 polypeptide. IL2 Activity may be measured, for example, in a cell proliferation assay using CTLL 2 mouse cytotoxic T cells, see Gearing, A. J. H. and C. B. Bird (1987) in Lymphokines and Interferons, A Practical Approach. Clemens, M. J. et al. (eds): IRL Press. 295. The specific activity of Recombinant Human IL2 is approximately 2.1×104 IU/μg, which is calibrated against recombinant human IL2 WHO International Standard (NIBSC code: 86/500). In some embodiments, for example when the IL2 orthogonal polypeptide of interest exhibits (or is engineered to possess) diminished affinity for CD25, IL2 activity may be assessed in human cells such as YT cells which do not require CD25 to provide signaling through the IL2 receptor but rather are capable of signaling through the intermediate affinity CD122/CD132 receptor. An orthogonal human IL2 of the present disclosure may have less than 20%, alternatively less than about 10%, alternatively less than about 8%, alternatively less than about 6%, alternatively less than about 4%, alternatively less than about 2%, alternatively less than about 1%, alternatively less than about 0.5% of the activity of WHO International Standard (NIBSC code: 86/500) wild-type mature human IL2 when evaluated at similar concentrations in a comparable assay.

The term “in need of treatment” as used herein refers to a judgment made by a physician or other caregiver with respect to a subject that the subject requires or will potentially benefit from treatment. This judgment is made based on a variety of factors that are in the realm of the physician's or caregiver's expertise.

As used herein the terms “intracellular domain of the modified CD122” or “ICD” refer to the portion of a transmembrane spanning orthogonal receptor that is inside of the plasma membrane of a cell expressing such transmembrane spanning orthogonal receptor. The ICD may comprise one or more “proliferation signaling domain(s)” or “PSD(s)” which refers to a protein domain which signals the cell to enter mitosis and begin cell growth. Examples include the Janus kinases, including but not limited to, JAK1, JAK2, JAK3, Tyk2, Ptk-2, homologous members of the Janus kinase family from other mammalian or eukaryotic species, the IL2 receptor β and/or γ chains and other subunits from the cytokine receptor superfamily of proteins that may interact with the Janus kinase family of proteins to transduce a signal, or portions, modifications or combinations thereof. Examples of signals include phosphorylation of one or more STAT molecules including but not limited to one or more of STAT1, STAT3, STAT5a, and/or STAT5b.

As used herein, the term “ligand” refers to a molecule that exhibits specific binding to a receptor and results in a change in the biological activity of the receptor so as to effect a change in the activity of the receptor to which it binds. In one embodiment, the term “ligand” refers to a molecule, or complex thereof, that can act as an agonist or antagonist of a receptor. As used herein, the term “ligand” encompasses natural and synthetic ligands. “Ligand” also encompasses small molecules, e.g., peptide mimetics of cytokines and peptide mimetics of antibodies. The complex of a ligand and receptor is termed a “ligand-receptor complex.”

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Substantial identity of amino acid sequences normally means sequence identity of at least 40%. Percent identity of polypeptides can be any integer from 40% to 100%, for example, at least 40%, 45%, 50%, 55%, 60%, 65%, 70%, 75%, 80%, 85%, 90%, 95%, or 99%. In some embodiments, polypeptides that are “substantially similar” share sequences as noted above except that residue positions that are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acids substitution groups are: valine-leucine-isoleucine, phenylalanine-tyrosine, lysine-arginine, alanine-valine, aspartic acid-glutamic acid, and asparagine-glutamine.

Algorithms that are suitable for determining percent sequence identity and sequence similarity are the BLAST and BLAST 2.0 algorithms, which are described in Altschul et al., Nuc. Acids Res. 25:3389-3402 (1977), and Altschul et al., J. Mol. Biol. 215:403-410 (1990), respectively. Software for performing BLAST analyses is publicly available on the Web through the National Center for Biotechnology Information (at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, (1989)) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” or “immediately C-terminal” refers to a position of a first amino acid residue relative to a second amino acid residue where the first and second amino acid residues are covalently bound to provide a contiguous amino acid sequence.

The terms “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

The term “operably linked” is used herein to refer to the relationship between nucleic acid sequences encoding differing functions when combined into a single nucleic acid sequence that, when introduced into a cell, provides a nucleic acid which is capable of effecting the transcription and/or translation of a particular nucleic acid sequence in a cell. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, certain genetic elements such as enhancers need not be contiguous with respect to the sequence to which they provide their effect.

As used herein the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences; fusion proteins with or without N-terminus methionine residues; fusion proteins with immunologically tagged proteins; fusion proteins of immunologically active proteins (e.g. antigenic diphtheria or tetanus toxin fragments) and the like.

As used herein the terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed due to genetic, experiential or environmental factors to having a particular disease, disorder or condition. In certain instances, the terms “prevent”, “preventing”, “prevention” are also used to refer to the slowing of the progression of a disease, disorder or condition from a present its state to a more deleterious state.

As used herein, the term “receptor” refers to a polypeptide having a domain that specifically binds a ligand that binding of the ligand results in a change to at least one biological property of the polypeptide. In some embodiments, the receptor is a “soluble” receptor that is not associated with a cell surface. The soluble form of hCD25 is an example of a soluble receptor that specifically binds hIL2. In some embodiments, the receptor is a cell surface receptor that comprises and extracellular domain (ECD) and a membrane associated domain which serves to anchor the ECD to the cell surface. In some embodiments of cell surface receptors, the receptor is a membrane spanning polypeptide comprising an intracellular domain (ICD) and extracellular domain (ECD) linked by a membrane spanning domain typically referred to as a transmembrane domain (TM). The binding of the ligand to the receptor results in a conformational change in the receptor resulting in a measurable biological effect. In some instances, where the receptor is a membrane spanning polypeptide comprising an ECD, TM and ICD, the binding of the ligand to the ECD results in a measurable intracellular biological effect mediated by one or more domains of the ICD in response to the binding of the ligand to the ECD. In some embodiments, a receptor is a component of a multi-component complex to facilitate intracellular signaling. For example, the ligand may bind a cell surface molecule having not associated with any intracellular signaling alone but upon ligand binding facilitates the formation of a heteromultimeric including heterodimeric (e.g. the intermediate affinity CD122/CD132 IL2 receptor), heterotrimeric (e.g. the high affinity CD25/CD122/CD132 hIL2 receptor) or homomultimeric (homodimeric, homotrimeric, homotetrameric) complex that results in the activation of an intracellular signaling cascade (e.g. the Jak/STAT pathway).

As used herein, the term “recombinant” or “engineered” to refer to polypeptides generated using recombinant DNA technology. The techniques and protocols for recombinant DNA technology are well known in the art.

The term “response,” for example, of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects.

As used herein the term “specifically binds” refers to the degree of selectivity or affinity for which one molecule binds to another. In the context of binding pairs (e.g. a ligand/receptor, antibody/antigen, antibody/ligand, antibody/receptor binding pairs) a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the first molecule of the binding pair when the affinity of the first molecule for the second molecule is at least two-fold greater, alternatively at least five times greater, alternatively at least ten times greater, alternatively at least 20-times greater, or alternatively at least 100-times greater than the affinity of the first molecule for other components present in the sample. In a particular embodiment, where the first molecule of the binding pair is an antibody, the antibody specifically binds to the second molecule of the binding pair (e.g. a protein, antigen, ligand, or receptor) if the equilibrium dissociation constant between antibody and to the second molecule of the binding pair is greater than about 106M, alternatively greater than about 108 M, alternatively greater than about 1010 M, alternatively greater than about 1011 M, alternatively greater than about 1010 M, greater than about 1012 M as determined by, e.g., Scatchard analysis (Munsen, et al. 1980 Analyt. Biochem. 107:220-239). In one embodiment where the ligand is an orthogonal IL2 and the receptor comprises an orthogonal CD122 ECD, the orthogonal IL2 specifically binds if the equilibrium dissociation constant of the IL2 ortholog/orthogonal CD122 ECD is greater than about 105M, alternatively greater than about 106 M, alternatively greater than about 107M, alternatively greater than about 108M, alternatively greater than about 109 M, alternatively greater than about 1010 M, or alternatively greater than about 1011 M. Specific binding may be assessed using techniques known in the art including but not limited to competition ELISA, BIACORE® assays and/or KINEXA® assays.

The terms “recipient”, “individual”, “subject”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is a human being.

As used herein the term “T-cell” or “T cell” is used in its conventional sense to refer to a lymphocytes that differentiates in the thymus, possess specific cell-surface antigen receptors, and include some that control the initiation or suppression of cell-mediated and humoral immunity and others that lyse antigen-bearing cells. In some embodiments the T cell includes without limitation naïve CD8+ T cells, cytotoxic CD8+ T cells, naïve CD4+ T cells, helper T cells, e.g. TH1, TH2, TH9, TH11, TH22, TFH; regulatory T cells, e.g. TR1, Tregs, inducible Tregs; memory T cells, e.g. central memory T cells, effector memory T cells, NKT cells, tumor infiltrating lymphocytes (TILs) and engineered variants of such T-cells including but not limited to CAR-T cells, recombinantly modified TILs and TCR engineered cells.

The phrase “therapeutically effective amount” as used herein in reference to the administration of an agent to a subject, either alone or as part of a pharmaceutical composition or treatment regimen, in a single dose or as part of a series of doses in an amount capable of having any detectable, positive effect on any symptom, aspect, or characteristic of a disease, disorder or condition when administered to the subject. The therapeutically effective amount can be ascertained by measuring relevant physiological effects, and it may be adjusted in connection with a dosing regimen and in response to diagnostic analysis of the subject's condition, and the like. The parameters for evaluation to determine a therapeutically effective amount of an agent are determined by the physician using art accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, biomarkers (such as inflammatory cytokines, IFN-□, granzyme, and the like), reduction in serum tumor markers, improvement in Response Evaluation Criteria In Solid Tumors (RECIST), improvement in Immune-Related Response Criteria (irRC), increase in duration of survival, extended duration of progression free survival, extension of the time to progression, increased time to treatment failure, extended duration of event free survival, extension of time to next treatment, improvement objective response rate, improvement in the duration of response, reduction of tumor burden, complete response, partial response, stable disease, and the like that that are relied upon by clinicians in the field for the assessment of an improvement in the condition of the subject in response to administration of an agent. As used herein the terms “Complete Response (CR),” “Partial Response (PR)” “Stable Disease (SD)” and “Progressive Disease (PD)” with respect to target lesions and the terms “Complete Response (CR),” “Incomplete Response/Stable Disease (SD)” and Progressive Disease (PD) with respect to non-target lesions are understood to be as defined in the RECIST criteria. As used herein the terms “immune-related Complete Response (irCR),” “immune-related Partial Response (irPR),” “immune-related Progressive Disease (irPD)” and “immune-related Stable Disease (irSD)” as as defined in accordance with the Immune-Related Response Criteria (irRC). As used herein, the term “Immune-Related Response Criteria (irRC)” refers to a system for evaluation of response to immunotherapies as described in Wolchok, et al. (2009) Guidelines for the Evaluation of Immune Therapy Activity in Solid Tumors: Immune-Related Response Criteria, Clinical Cancer Research 15(23): 7412-7420. A therapeutically effective amount may be adjusted over a course of treatment of a subject in connection with the dosing regimen and/or evaluation of the subject's condition and variations in the foregoing factors. In one embodiment, a therapeutically effective amount is an amount of an agent when used alone or in combination with another agent does not result in non-reversible serious adverse events in the course of administration to a mammalian subject.

The terms “treat”, “treating”, treatment” and the like refer to a course of action (such as administering IL2, a CAR-T cell, or a pharmaceutical composition comprising same) initiated with respect to a subject after a disease, disorder or condition, or a symptom thereof, has been diagnosed, observed, or the like in the subject so as to eliminate, reduce, suppress, mitigate, or ameliorate, either temporarily or permanently, at least one of the underlying causes of such disease, disorder, or condition afflicting a subject, or at least one of the symptoms associated with such disease, disorder, or condition. The treatment includes a course of action taken with respect to a subject suffering from a disease where the course of action results in the inhibition (e.g., arrests the development of the disease, disorder or condition or ameliorates one or more symptoms associated therewith) of the disease in the subject.

The terms “regulatory T cell” or “Treg cell” as used herein refers to a type of CD4+ T cell that can suppress the responses of other T cells including but not limited to effector T cells (Teff). Treg cells are characterized by expression of CD4, the a-subunit of the IL2 receptor (CD25), and the transcription factor forkhead box P3 (FOXP3) (Sakaguchi, Annu Rev Immunol 22, 531-62 (2004). By “conventional CD4+ T cells” is meant CD4+ T cells other than regulatory T cells.

Wild Type: By “wild type” or “WT” or “native” herein is meant an amino acid sequence or a nucleotide sequence that is found in nature, including allelic variations. A WT protein, polypeptide, antibody, immunoglobulin, IgG, etc. has an amino acid sequence or a nucleotide sequence that has not been modified by the hand of man.

### Modified Human CD122

The present disclosure provides modified human CD122 comprising, in addition to a native STAT5 recognition motif, one or more STAT3 binding motifs. Having the additional STAT3 binding motifs boosts the signaling and also stabilizes the IL2 response.

### STAT Proteins and STAT3 Binding Motifs

STAT proteins act as transcriptional activators upon phosphorylation of a conserved tyrosine residue at the C terminus followed by translocation into the nucleus, where they bind to DNA and activate target gene transcription. Hennighausen L, Robinson G W. Genes Dev. 2008; 22:711-21. There are seven STAT proteins in the family: STAT1, STAT2, STAT3, STAT4, STAT5A, STAT5B and STAT6, and they have functions in a variety of pathways, from innate and acquired immunity to cell proliferation, differentiation and survival. Basham et al., Nucleic Acids Res. 2008 June; 36(11): 3802-3818.

STAT binding motifs are typically present in cytokine receptors, and binding of their respective cytokines activates the tyrosine kinases in the Janus kinease (JAK) families, which phorphorylates certain tyrosines in the intracellular domain of the receptors. The phorphorylated receptor recruits STATs to STAT recognition motifs on the receptor and become phorphorylated. The phorphorylated STATs are dimerized and translocated into the nucleus and activate transcription of important genes, Hennighausen L, Robinson G W. Interpretation of cytokine signaling through the transcription factors STAT5A and STAT5B. Genes Dev. 2008; 22:711-21.

Of these STAT proteins, STAT5 can be activated by cytokines including IL2, IL-4, IL-7, IL-9, IL-15, and IL21 through binding to their cognate receptors. Lara E. Kallal & Christine A. Biron (2013) Changing partners at the dance, JAK-STAT, 2:1, e23504, DOI: 10.4161/jkst.23504, Page 2, Col. 2. Activated STAT5 can target genes scuh as recognition motif and recruit STAT5, which activated the transcription of such as Cis, spi2.1, and Socs-1. Basham et al., Nucleic Acids Res. 2008 June; 36(11): 3802-3818. For example, a modified CD122 of this disclosure, aka the beta receptor for IL2, contains STAT5 recognition motif and can recruit and activate STAT5.

The STAT5 motif has a sequence of YX1X2L ((SEQ ID NO: 19). X1 and X2 can be any natural amino acid. In some cases, X1 and X2 are the same amino acid residues. In some cases, X1 and X2 are different amino acid residues. In one embodiment, the STAT5 has a sequence of YLSL (SEQ ID NO: 20).

The STAT3 binding motif is not present in native human CD122. It is typically present in receptors that bind to IL-6, IL-10, IL21, IFNalpha beta, IFN gamma, and IFN lambda. Upon activation, STAT3 target, Bcl-XL, survivin, cyclin D1, and activating c-myc. Lara E. Kallal & Christine A. Biron (2013) Changing partners at the dance, JAK-STAT, 2:1, e23504, DOI: 10.4161/jkst.23504, page 3. STAT3 can be activated through tyrosine phosphorylation by a variety of cytokines whose receptors share the gp130 chain, including IL-6 and IL21, oncostatin M (OSM) and leukemia inhibitory factor (LIF) [2]. STAT3 has roles in a variety of biological functions including oncogenesis, angiogenesis and tumor metastasis, and, anti-apoptosis. See Wei Sun et al., FEBS Lett. 2006 Oct. 30;580(25):5880-4. Epub 2006 Oct. 2. and Fukada et al. Immunity, 1 Nov. 1996, 449-460 Vol. 5, issue 5. Thus, STAT3 signaling confer the cells anti-apoptotic properties and thus increase half life of cells expressing the modified human CD122.

In this disclosure, a human CD122 (comprising the intact STAT5 motif) has been modified to introduce one or more STAT3 binding motifs and the modified human CD122 so produced retains STAT5 recognition motif and gains one or more STAT3 binding motifs.

In some embodiments, the modified CD122 may comprise one, two, three, or more STAT3 binding motifs. STAT3 recognition motif has a sequence of YX1X2Q (SEQ ID NO: 21). In some embodiments, X1 is selected from the group consisting of L, R, F, M, and X2 is selected from the group consisting of R, K, H, and P. In some embodiments, the STAT3 sequence that is selected from the group consisting of: YLRQ (SEQ ID NO:11); YLKQ (SEQ ID NO: 12); YRHQ (SEQ ID NO: 13); YLRQ (SEQ ID NO: 14); YFKQ (SEQ ID NO: 15); YLPQ (SEQ ID NO: 16); YMPQ (SEQ ID NO: 17), and YDKPH (SEQ ID NO: 18).

### Human CD122 (Including Native Human CD122 and Human CD122 Ortholog)

In addition to the STAT3 binding motifs, the modified human CD122 comprises a human CD122, which can be a native human CD122 or a human orthogonal CD122.

In some embodiments, the modified human CD122 comprises an orthogonal human CD122. The orthogonal human CD122 is produced by mutating residues of native CD122 such that they specifically bind to an orthogonal IL2 but does not specifically bind to a native IL2. For example the binding affinity to the orthogonal IL2 is higher, e.g., 2×, 3×, 4×, 5×, 10× or more of the affinity of the native IL2 for the native CD122. In some embodiments, the affinity of the orthogonal IL2 for the cognate orthogonal CD122 exhibits affinity comparable to the affinity of the native IL2 for the native CD122, e.g., having an affinity that is least about 1% of the binding affinity of the native CD122 for the native IL2, at least about 5%, at least about 10%, at least about 25%, at least about 50%, at least about 75%, at least about 100%. In some cases, the orthogonal CD122 is modified at one or more residues selected from R41, R42, Q70, K71, T73, T74, V75, 5132, H133, Y134, F135, E136, and Q214 relative to native human CD122. In some embodiments, the orthogonal human CD122 is modified at H133 and Y134. In some embodiments, the orthogonal human CD122 comprises substitutions of H133D and Y134F. In some embodiments, the orthogonal human CD122 comprises amino acid substitutions at Q70, T73, H133, and Y134 relative to the native human CD122 protein. In some embodiments, orthogonal human CD122 comprises amino acid substitutions H133 and Y134. In some embodiments the amino acid substitution is made to an acidic amino acid, e.g., aspartic acid and/or glutamic acid. Specific amino acid substitutions include, without limitation, Q70Y; T73D; T73Y; H133D, H133E; H133K; Y134F; Y134E; Y134R relative to the native human CD122. The selection of an orthologous cytokine may vary with the choice of orthologous receptor.

In some embodiments, in addition to having the above substitutions relative to the native human CD122, the orthogonal human CD122 has a sequence that is at least 90%, at least 91%, at least 92%, at least 93%, at least 94%, at least 95%, at least 96%, at least at least 97%, at least 98% or at least 99% identical to the full length sequence of the native CD122 (SEQ ID NO: 2).

### Orthogonal IL2:

In cases where a modified orthogonal human CD122 is used, an orthogonal IL2 may be used to facilitate selective stimulation of cells engineered to express the modified orthogonal CD122. The contact of engineered T cells that express the modified orthogonal CD122 with a corresponding orthogonal ligand for such orthogonal CD122 enables specific activation of such engineered T cells. In particular this orthogonal IL2 receptor ligand complex provides for selective expansion of cells engineered to express the orthogonal receptor in a mixed population of cells, in particular a mixed population of T cells. The IL2 orthogonal ligands that provide selective binding and signaling via receptors comprising the extracellular domain of a CD122 orthogonal receptor, in particular the extracellular domain of human CD122 comprising the amino acid substitutions H133D and Y134F. The IL2 activity of the orthogonal IL2 on cells expressing the wild-type CD122 is significantly diminished compared to the activity orthogonal IL2 on cells expressing the orthogonal CD122. Thus, selective activation and/or expansion of engineered cells expressing a receptor comprising the extracellular domain of orthogonal IL2 using orthogonal IL2 on the engineered cell populations is provided.

An orthogonal IL2 incorporates modifications to its primary structure to provide polypeptide variant that exhibits: (a) significantly reduced affinity to its native CD122 (i.e., the native receptor for the native IL2 from which the orthogonal IL2 is derived); and (b) specific binding a engineered orthogonal CD122 which is a variant of native CD122. Upon binding of the orthogonal IL2 to the orthogonal CD122 (which is expressed on surface of cell which has been modified by recombinant DNA technology to incorporate a nucleic acid sequence encoding the orthogonal receptor operably linked to control elements to effect the expression of the orthogonal receptor in the recombinantly modified cell), the activated orthogonal CD122 initiates signaling that is transduced through native cellular elements to provide for a biological activity that mimics that native response of the cognate but which is specific to the recombinantly modified cell population expressing the orthogonal receptor. In some embodiments of the invention, orthologs possess significant selectivity for the orthogonal CD122 relative to the native CD122 receptor and optionally possessing significantly reduced potency with respect to the native CD122. Selectivity is typically assessed by activity measured in an assay characteristic of the activity induced in response to ligand/receptor binding. In some embodiments, the ortholog IL2 possesses at least 5 fold, alternatively at least 10 fold, alternatively at least 20 fold, alternatively at least 30 fold, alternatively at least 40 fold, alternatively at least 50 fold, alternatively at least 100 fold, alternatively at least 200 fold difference in EC50 as measured in the same assay.

The IL2 orthologs exhibit specific binding for the extracellular domain of an ortholog CD122, e.g., a human CD122 that incorporates modifications at position 133 and 134.

### IL2 Orthologs

In various embodiments, methods of the present disclosure comprise the use of orthogonal IL2s comprising an amino acid sequence of the following formula 1:
(AA1)-(AA2)-(AA3)-(AA4)-(AA5)-(AA6)-(AA7)-(AA8)-(AA9)i-T10-Q11-L12-(AA13)-(AA14)-(AA15)-(AA16)-L17-(AA18)-(AA19)-(AA20)-L21-(AA22)-(AA23)-I24-L25-N26-(AA27)-I28-N29-N30-Y31-K32-N33-P34-K35-L36-T37-(AA38)-(AA39)-L40-T41-(AA42)-K43-F44-Y45-M46-P47-K48-K49-A50-(AA51)-E52-L53-K54-(AA55)-L56-Q57-C58-L59-E60-E61-E62-L63-K64-P65-L66-E67-E68-V69-L70-N71-L72-A73-(AA74)-S75-K76-N77-F78-H79-(AA80-(AA81)-P82-R83-D84-(AA85)-(AA86)-S87-N88-(AA89)-N90-(AA91)-(AA92)-V93-L94-E95-L96-(AA97)-G98-S99-E100-T101-T102-F103-(AA104)-C105-E106-Y107-A108-(AA109)-E110-T111-A112-(AA113)-I114-V115-E116-F117-L118-N119-R120-W121-I122-T123-F124-(AA125)-(AA126)-S127-I128-I129-(AA130)-T131-L132-T133
   wherein:

AA1 is A (wild type) or deleted;

AA2 is P (wild type) or deleted;

AA3 is T (wild type), C, A, G, Q, E, N, D, R, K, P, or deleted

AA4 is S (wild type) or deleted;

AA5 is S (wild type) or deleted;

AA6 is S (wild type) or deleted;

AA7 is T (wild type) or deleted;

AA8 is K (wild type) or deleted;

AA9 is K (wild type) or deleted;

AA13 is Q (wild type), W or deleted;

AA14 is L (wild type), M, W or deleted;

AA15 is E (wildtype), K, D, T, A, S, Q, H or deleted;

AA16 is H (wildtype), N or Q or deleted;

AA18 is L (wild type) or R, L, G, M, F, E, H, W, K, Q, S, V, I, Y, H, D or T;

AA19 is L (wildtype), A, V, I or deleted;

AA20 is D (wildtype), T, S M L, or deleted;

AA22 is Q (wild type) or F, E, G, A, L, M, F, W, K, S, V, I, Y, H, R, N, D, T, F or deleted

AA23 is M (wild type), A, W, H, Y, F, Q, S, V, L, T, or deleted;

AA27 IS G (wildtype), K, S or deleted;

AA38 is R (wild type), W or G;

AA39 is M (wildtype), L or V;

AA42 is F (wildtype) or K;

AA51 is T (wildtype), I or deleted

AA55 is H (wildtype) or Y;

AA74 is Q (wild type), N, H, S;

AA80 is L (wild type), F or V;

AA81 is R (wild type), I, D, Y, T or deleted

AA85 is L (wild type) or V;

AA86 is I (wild type) or V;

AA88 is N (wildtype), E or Q or deleted;

AA89 is I (wild type) or V;

AA91 is V (wild type), R or K;

AA92 is I (wild type) or F;

AA97 is K (wild type) or Q;

AA104 is M (wild type) or A;

AA109 is D (wildtype), C or a non-natural amino acid with an activated side chain;

AA113 is T (wild type) or N;

AA125 is C (wild type), A or S;

AA126 is Q (wild type) or H, M, K, C, D, E, G, I, R, S, or T; and/or

AA130 is S (wild type), T or R.

In some embodiments, the present disclosure provides orthogonal IL2s which are hIL2 polypeptides comprising the following sets of amino acid modifications:

[E15S-H16Q-L19V-D20L-Q22K]

[H16N, L19V, D20N, Q22T, M23H, G27K];

[E15D, H16N, L19V, D20L, Q22T, M23H];

[E15D, H16N, L19V, D20L, Q22T, M23A],

[E15D, H16N, L19V, D20L, Q22K, M23A];

[E15S; H16Q; L19V, D20T; Q22K, M23L];

[E15S; H16Q; L19V, D20T; Q22K, M23S];

[E15S; H16Q; L19V, D20S; Q22K, M23S];

[E15S; H16Q; L19I, D20S; Q22K; M23L];

[E15S; L19V; D20M; Q22K; M23S];

[E15T; H16Q; L19V; D20S; M23S];

[E15Q; L19V; D20M; Q22K; M23S];

[E15Q; H16Q; L19V; D20T; Q22K; M23V];

[E15H; H16Q; L19I; D20S; Q22K; M23L];

[E15H; H16Q; L19I; D20L; Q22K; M23T];

[L19V; D20M; Q22N; M23S].

### Cys125:

In some embodiments, the present disclosure provides orthogonal IL2s to facilitate recombinant expression in bacterial cells by eliminating the unpaired cysteine residue at position 125 and/or elimination of the N-terminal Met of the directly expressed IL2 polypeptide as well as the alanine at position 1 by post-translational processing by endogenous bacterial proteases. When an amino acid is missing, it is referred to as “des”. In some embodiments, the cysteine at position 125 is substituted with alanine or serine (C125A or C125S). Such mutations are typically used to avoid misfolding of the protein when expressed recombinantly in bacteria and isolated from inclusion bodies. For example, “des-Ala1” means the alanine as position 1 is absent in the IL2 polypeptide. In some embodiments, the orthogonal IL2s or the present invention comprise one of the following sets of amino acid modifications:

[E15S-H16Q-L19V-D20L-Q22K-M23A-C125S];

[E15S-H16Q-L19V-D20L-Q22K-C125S];

[E15S-H16Q-L19V-D20L-M23A-C125S];

[E15S-H16Q-L19V-D20L-C1255];

[E15S-H16Q-L19V-D20L-Q22K-M23A-C125A];

[E15S-H16Q-L19V-D20L-M23A-C125A];

[E15S-H16Q-L19V-D20L-Q22K-C125A];

[E15S-H16Q-L19V-D20L-C125A];

[desAla1-E15S-H16Q-L19V-D20L-Q22K-M23A-C125S];

[desAla1-E15S-H16Q-L19V-D20L-Q22K-C125S];

[desAla1-E15S-H16Q-L19V-D20L-C125S];

[desAla1-E15S-H16Q-L19V-D20L-Q22K-M23A-C125A];

[desAla1-E15S-H16Q-L19V-D20L-Q22K-C125A];

[desAla1-E15S-H16Q-L19V-D20L-C125A];

[desAla1-E15S-H16Q-L19V-D20L-Q22K-M23A];

[desAla1-E15S-H16Q-L19V-D20L-M23A];

[desAla1-E15S-H16Q-L19V-D20L-Q22K]; or

[desAla1-E15S-H16Q-L19V-D20L].

### Mutations to Increase CD122 Affinity

In some embodiments, orthogonal IL2s contain one or more mutations in positions of the hIL2 sequence that either contact hCD122 or alter the orientation of other positions contacting CD122, resulting in an orthogonal IL2 having increased affinity for CD122. IL2 residues that have been identified as being involved in the binding of IL2 to CD122 include L12, Q13, H16, L19, D20, M23, Q74, L80, R81, D84, L85, 186, S87, N88, 189 V91, 192, and E95. In some embodiments, the orthogonal IL2 comprises one or more of the amino acid substitutions: Q74N, Q74H, Q74S, L80F, L80V, R81D, R81T, L85V, I86V, I89V, and/or I92F or combinations thereof. In some embodiments, the orthogonal IL2 comprises one or more of the amino acid substitutions: L80F, R81D, L85V, I86V and I92F. In some embodiments, the orthogonal IL2 comprises one or more of the amino acid substitutions: N74Q, L80F, R81D, L85V, I86V, I89V, and I92F. In some embodiments, the orthogonal IL2 comprises one or more of the amino acid substitutions: Q74N, L80V, R81T, L85V, I86V, and I92F. In some embodiments, the orthogonal IL2 comprises one or more of the amino acid substitutions: Q74H, L80F, R81D, L85V, I86V and I92F. In some embodiments, the orthogonal IL2 comprises one or more of the amino acid substitutions: Q74S, L80F, R81D, L85V, I86V and I92F. In some embodiments, the orthogonal IL2 comprises one or more of the amino acid substitutions: Q74N, L80F, R81D, L85V, I86V and I92F. In some embodiments, the orthogonal IL2 comprises one or more of the amino acid substitutions: Q74S, R81T, L85V, and I92F. In some embodiments, the orthogonal IL2 comprises [L80E-R81D-L85V-I86V-I92F]. In some embodiments, the present disclosure provides orthogonal IL2s which comprise one of the following sets of amino acid modifications:

[E15S-H16Q-L19V-D20L-M23A-L80E-R81D-L85V-I86V-I92F];

[E15S-H16Q-L19V-D20L-Q22K-L80E-R81D-L85V-I86V-I92F];

[E15S-H16Q-L19V-D20L-Q22K-M23A L80E-R81D-L85V-I86V-I92F];

[E15S-H16Q-L19V-D20L-M23A-L80E-R81D-L85V-I86V-I92F-Q126H];

[E15S-H16Q-L19V-D20L-Q22K-L80E-R81D-L85V-I86V-I92F-Q126H];

[E15S-H16Q-L19V-D20L-Q22K-M23A-L80E-R81D-L85V-I86V-I92F-Q126H];

[E15S-H16Q-L19V-D20L-M23A-L80E-R81D-L85V-I86V-I92F-Q126M];

[E15S-H16Q-L19V-D20L-Q22K-L80E-R81D-L85V-I86V-I92F-Q126M]; or

[E15S-H16Q-L19V-D20L-Q22K-M23A-L80E-R81D-L85V-I86V-I92F-Q126M].

In some embodiments, the orthologs comprise the substitution L85V that has been identified as increasing affinity of IL2 to CD122. In some embodiments, the present disclosure provides orthogonal IL2s which are hIL2 polypeptides comprising one of the following sets of amino acid modifications:

[E15S-H16Q-L19V-D20L-M23A-L85V];

[E15S-H16Q-L19V-D20L-Q22K-M23A-L85V];

[E15S-H16Q-L19V-D20L-M23A-L85V];

[E15S-H16Q-L19V-D20L-Q22K-M23A-L85V];

[E15S-H16Q-L19V-D20L-M23A-L85V-Q126H];

[E15S-H16Q-L19V-D20L-Q22K-M23A-L85V-Q126H];

[E15S-H16Q-L19V-D20L-M23A-L85V-Q126M]; or

[E15S-H16Q-L19V-D20L-Q22K-M23A-L85V-Q126M].

### Modulating CD25 Affinity

In some embodiments, the orthogonal IL2s contain one or more mutations in positions of the IL2 sequence that either contact CD25 or alter the orientation of other positions contacting CD25 resulting in a decreased affinity for CD25. The mutations may be in or near areas known to be in close proximity to CD25 based on published crystal structures (Wang, et al Science 310:1159 2005). IL2 residues believed to contact CD25 include K35, R38, T41, F42, K43, F44, Y45, E61, E62, K64, P65, E68, V69, L72, and Y107. In some embodiments, the orthogonal IL2s of the present disclosure comprise one or more of the point mutations of R38A, F41A and F42A (Suave, et al (1991) PNAS (USA) 88:4636-4640); P65L (Chen et al. Cell Death and Disease (2018) 9:989); F42A/G/S/T/Q/E/N/R/K, Y45A/G/S/T/Q/E/N/D/R/K/ and/or L72G/A/S/T/Q/E/N/D/R/K (Ast, et al United States Patent Application Publication 2012/0244112A1 published Sep. 27, 2012; U.S. Pat. No. 9,266,938B2 issued Feb. 23, 2016). Particular combinations of substitutions have been identified as reducing binding to CD25. In some embodiments, the orthogonal IL2s of the present disclosure comprise one or more of the of the sets of substitutions [R38A-F42A-Y45A-E62A] as described in Carmenate, et al (2013) J Immunol 190:6230-6238; [F42A-Y45A-L72G] (Roche RG7461 (R06874281); and/or [T41P-T51P] (Chang, et al (1995) Molecular Pharmacology 47:206-211). In some embodiments, the present disclosure provides orthogonal IL2s which are hIL2 polypeptides comprising one of the following sets of amino acid modifications:

[E15S-H16Q-L19V-D20L-M23A-R38A-F42A-Y45A-E62A];

[E15S-H16Q-L19V-D20L-M23A-R38A-F42A-Y45A-E62A];

[E15S-H16Q-L19V-D20L-Q22K-M23A-R38A-F42A-Y45A-E62A];

[E15S-H16Q-L19V-D20L-M23A-R38A-F42A-Y45A-E62A-Q126H];

[E15S-H16Q-L19V-D20L-M23A-R38A-F42A-Y45A-E62A-Q126H];

[E15S-H16Q-L19V-D20L-Q22K-M23A-R38A-F42A-Y45A-E62A-Q126H];

[E15S-H16Q-L19V-D20L-M23A-R38A-F42A-Y45A-E62A-Q126M]; or

[E15S-H16Q-L19V-D20L-M23A-R38A-F42A-Y45A-E62A-Q126M].

In some embodiments of the invention, the orthogonal IL2s contain one or more mutations in positions of the IL2 sequence that either contact CD132 or alter the orientation of other positions contacting CD132 resulting in an altered binding to CD132. Exemplary orthogonal IL2s contain one or more mutations in positions of the IL2 sequence that either contact CD132 or alter the orientation of other positions contacting CD122, resulting in an altered binding to CD132. IL2 residues believed to contact CD132 include Q11, L18, Q22, E110, N119, T123, Q126, 5127, 1129, 5130, and T133. In some embodiments, the IL2 comprises modifications at L18 AA18 is L (wild type) or R, L, G, M, F, E, H, W, K, Q, S, V, I, Y, H, D or T; AA126 is Q (wild type) or H, M, K, C, D, E, G, I, R, S, or T; and/or AA22 is Q (wild type) or F, E, G, A, L, M, F, W, K, S, V, I, Y, H, R, N, D, T, or F.

In some embodiments, the present disclosure provides orthogonal IL2s which are hIL2 polypeptides comprising one the following sets of amino acid modifications:

[E15S-H16Q-L19V-D20L-M23A-Q126H];

[E15S-H16Q-L19V-D20L-Q22K-M23A-Q126H];

[E15S-H16Q-L19V-D20L-Q22K-Q126H];

[E15S-H16Q-L19V-D20L-M23A-Q126M];

[E15S-H16Q-L19V-D20L-Q22K-M23A-Q126M];

[E15S-H16Q-L19V-D20L-Q22K-Q126M];

[desAla1-E15S-H16Q-L19V-D20L-Q126M];

[desAla1-E15S-H16Q-L19V-D20L-Q22K-Q126M];

[desAla1-E15S-H16Q-L19V-D20L-M23A-Q126M];

[desAla1-E15S-H16Q-L19V-D20L-Q22K-M23A-Q126M];

[desAla1-E15S-H16Q-L19V-D20L-Q22K-M23A-Q126M];

[E15S-H16Q-L19V-D20L-M23A-L80E-R81D-I86V-I92F-Q126H];

[E15S-H16Q-L19V-D20L-Q22K-L80E-R81D-I86V-I92F-Q126H];

[E15S-H16Q-L19V-D20L-Q22K-M23A-L80E-R81D-I86V-I92F-Q126H];

[E15S-H16Q-L19V-D20L-M23A-L80E-R81D-I86V-I92F-Q126M];

[E15S-H16Q-L19V-D20L-Q22K-M23A-L80E-R81D-I86V-I92F-Q126M];

[E15S-H16Q-L19V-D20L-M23A-L85V-Q126H];

[E15S-H16Q-L19V-D20L-Q22K-L85V-Q126H];

[E15S-H16Q-L19V-D20L-Q22K-M23A-L85V-Q126H];

[E15S-H16Q-L19V-D20L-M23A-L85V-Q126M];

[E15S-H16Q-L19V-D20L-Q22K-L85V-Q126H]; or

[E15S-H16Q-L19V-D20L-Q22K-M23A-L85V-Q126M].

When produced recombinantly in bacterial expression systems directly in the absence of a leader sequence, endogenous proteases result in the deletion of the N-terminal Met-Ala1 residues to provide “desAla1” orthogonal IL2s. In some embodiments, the present disclosure provides orthogonal IL2s which are hIL2 polypeptides comprising one of the following sets of amino acid modifications:

[desAla1-E15S-H16Q-L19V-D20L];

[desAla1-E15S-H16Q-L19V-D20L-Q22K];

[desAla1-E15S-H16Q-L19V-D20L-Q22K-M23A];

[desAla1-E15S-H16Q-L19V-D20L-Q126H];

[desAla1-E15S-H16Q-L19V-D20L-Q22K-Q126H];

[desAla1-E15S-H16Q-L19V-D20L-Q22K-M23A-Q126H];

[desAla1-E15S-H16Q-L19V-D20L-Q126M];

[desAla1-E15S-H16Q-L19V-D20L-Q22K-Q126H];

[desAla1-E15S-H16Q-L19V-D20L-Q22K-M23A-Q126H];

[desAla1-E15S-H16Q-L19V-D20L-C125A];

[desAla1-E15S-H16Q-L19V-D20L-Q22K-C125A];

[desAla1-E15S-H16Q-L19V-D20L-Q22K-M23A-C125A];

[desAla1-E15S-H16Q-L19V-D20L-C125A-Q126H];

[desAla1-E15S-H16Q-L19V-D20L-Q22K-C125A-Q126H];

[desAla1-E15S-H16Q-L19V-D20L-Q22K-M23A-C125A-Q126H];

[desAla1-E15S-H16Q-L19V-D20L-C125A-Q126M];

[desAla1-E15S-H16Q-L19V-D20L-Q22K-C125A-Q126H];

[desAla1-E15S-H16Q-L19V-D20L-Q22K-M23A-C125A-Q126H];

[desAla1-E15S-H16Q-L19V-D20L-C125S];

[desAla1-E15S-H16Q-L19V-D20L-Q22K-C125S];

[desAla1-E15S-H16Q-L19V-D20L-Q22K-M23A-C125S];

[desAla1-E15S-H16Q-L19V-D20L-C125S-Q126H];

[desAla1-E15S-H16Q-L19V-D20L-Q22K-C125S-Q126H];

[desAla1-E15S-H16Q-L19V-D20L-Q22K-M23A-C125S-Q126H];

[desAla1-E15S-H16Q-L19V-D20L-C125S-Q126M];

[desAla1-E15S-H16Q-L19V-D20L-Q22K-C125S-Q126M]; or

[desAla1-E15S-H16Q-L19V-D20L-Q22K-M23A-C125S-Q126M].

### Conservative Amino Acid Substitutions

In addition to the foregoing modifications that contribute to the activity and selectivity of the orthogonal IL2 for the CD122 orthogonal receptor, the orthogonal IL2 may comprise one or more modifications to its primary structure that provide minimal effects on the activity IL2. In some embodiments, the orthogonal IL2s of the present disclosure may further comprise one more conservative amino acid substitution within the wild type IL-2 amino acid sequence. Such conservative substitutions include those described by Dayhoff in The Atlas of Protein Sequence and Structure 5 (1978), and by Argos in EMBO J., 8:779-785 (1989). Conservative substitutions are generally made in accordance with the following chart depicted as Table 3.

TABLE 3
Exemplary Conservative Amino Acid Substitutions

| Wild type Residue | Substitution(s) |
|---|---|
| Ala | Ser |
| Arg | Lys |
| Asn | Gln, His |
| Asp | Glu |
| Cys | Ser, Ala |
| Gln | Asn |
| Glu | Asp |
| Gly | Pro |
| His | Asn, Gln |
| Ile | Leu, Val |
| Leu | Ile, Val |
| Lys | Arg, Gln, Glu, Met, Leu, Ile |
| Phe | Met, Leu, Tyr, Trp |
| Ser | Thr |
| Thr | Ser |
| Trp | Tyr, Phe |
| Tyr | Trp, Phe |
| Val | Ile, Leu |

Substantial changes in function or immunological identity may be made by selecting amino acid substitutions that are less conservative than those indicated in Table 3. For example substitutions may be made which more significantly affect the structure of the polypeptide backbone or disrupt secondary or tertiary elements including the substitution of an amino acid with a small uncharged side chain (e.g. glycine) with a large charge bulky side chain (asparagine). In particular, substitution of those IL2 residues which are involved in the amino acids that interact with one or more of CD25, CD122 and/or CD123 as may be discerned from the crystal structure of IL2 in association with its receptors as described in

In addition to the foregoing modifications that contribute to the activity and selectivity of the orthogonal IL2 for the CD122 orthogonal receptor, the orthogonal IL2 may comprise one or more modifications to its primary structure. Modifications to the primary structure as provided above may optionally further comprise modifications do not substantially diminish IL2 activity of the orthogonal IL2 including but not limited to the substitutions: N30E; K32E; N33D; P34G; T37I, M39Q, F42Y, F44Y, P47G, T51I, E52K, L53N, Q57E, M104A (see U.S. Pat. No. 5,206,344).

### Removal of Glycosylation Site

The orthogonal IL2s of the present disclosure may comprises comprise modifications to eliminate the O-glycosylation site at position Thr3 of the to facilitate the production of an aglycosylated orthogonal IL2 when the orthogonal IL2 expressed in mammalian cells such as CHO or HEK cells. Thus, in certain embodiments the orthogonal IL2 comprise a modification which eliminates the O-glycosylation site of IL-2 at a position corresponding to residue 3 of human IL-2. In one embodiment said modification which eliminates the O-glycosylation site of IL-2 at a position corresponding to residue 3 of human IL-2 is an amino acid substitution. Exemplary amino acid substitutions include T3A, T3G, T3Q, T3E, T3N, T3D, T3R, T3K, and T3P which removes the glycosylation site at position 3 without eliminating biological activity (see U.S. Pat. No. 5,116,943; Weiger et al., (1989) Eur. J. Biochem., 180:295-300). In a specific embodiment, said modification is the amino acid substitution T3A. In some embodiments, the present disclosure provides orthogonal IL2s which are hIL2 polypeptides comprising one of the following sets of amino acid modifications:

[T3A-E15S-H16Q-L19V-D20L-Q22K-M23A-C125S];

[T3A-E15S-H16Q-L19V-D20L-Q22K-C125S];

[T3A-E15S-H16Q-L19V-D20L-M23A-C125S];

[T3A-E15S-H16Q-L19V-D20L-C125S];

[T3A-E15S-H16Q-L19V-D20L-Q22K-M23A-C125A];

[T3A-E15S-H16Q-L19V-D20L-M23A-C125A];

[T3A-E15S-H16Q-L19V-D20L-Q22K-C125A];

[T3A-E15S-H16Q-L19V-D20L-C125A];

[T3A-E15S-H16Q-L19V-D20L-Q22K-M23A];

[T3A-E15S-H16Q-L19V-D20L-M23A];

[T3A-E15S-H16Q-L19V-D20L-Q22K];

[T3A-E15S-H16Q-L19V-D20L];

[desAla1-T3A-E15S-H16Q-L19V-D20L-Q22K-M23A-C125S];

[desAla1-T3A-E15S-H16Q-L19V-D20L-M23A-C125S];

[desAla1-T3A-E15S-H16Q-L19V-D20L-Q22K-C125S];

[desAla1-T3A-E15S-H16Q-L19V-D20L-C125S];

[desAla1-T3A-E15S-H16Q-L19V-D20L-Q22K-M23A-C125A];

[desAla1-T3A-E15S-H16Q-L19V-D20L-M23A-C125A];

[desAla1-T3A-E15S-H16Q-L19V-D20L-Q22K-C125A];

[desAla1-T3A-E15S-H16Q-L19V-D20L-C125A];

[desAla1-T3A-E15S-H16Q-L19V-D20L-Q22K-M23A];

[desAla1-T3A-E15S-H16Q-L19V-D20L-M23A];

[desAla1-T3A-E15S-H16Q-L19V-D20L-Q22K]; or

[desAla1-T3A-E15S-H16Q-L19V-D20L].

Orthogonal IL2s may comprise deletion of the first two amino acids (desAla1-desPro2) as well as substitution of the Thr3 glycosylation with a cysteine residue to facilitate for selective N-terminal modification, especially pegylation of the sulfhydryl group of the cysteine (See, e.g., Katre, et al. U.S. Pat. No. 5,206,344 issued Apr. 27, 1993). In some embodiments, the present disclosure provides orthogonal IL2s which are hIL2 polypeptides comprising one of the following sets of amino acid modifications:

[desAla1-desPro2-T3C-E15S-H16Q-L19V-D20L-Q22K-M23A-C125S];

[desAla1-desPro2-T3C-E15S-H16Q-L19V-D20L-Q22K-C125S];

[desAla1-desPro2-T3C-E15S-H16Q-L19V-D20L-M23A-C125S];

[desAla1-desPro2-T3C-E15S-H16Q-L19V-D20L-C125S];

[desAla1-desPro2-T3C-E15S-H16Q-L19V-D20L-Q22K-M23A-C125A];

[desAla1-desPro2-T3C-E15S-H16Q-L19V-D20L-Q22K-C125A];

[desAla1-desPro2-T3C-E15S-H16Q-L19V-D20L-M23A-C125A];

[desAla1-desPro2-T3C-E15S-H16Q-L19V-D20L-C125A];

[desAla1-desPro2-T3C-E15S-H16Q-L19V-D20L-Q22K-M23A];

[desAla1-desPro2-T3C-E15S-H16Q-L19V-D20L-Q22K];

[desAla1-desPro2-T3C-E15S-H16Q-L19V-D20L-M23A]; or

[desAla1-desPro2-T3C-E15S-H16Q-L19V-D20L].

### Oxidation Stabilized M104A

An orthogonal IL2 disclosed herein may optionally further comprise a modification at position M104, in one embodiment the substitution of methionine 104 with an alanine residue (M104A) to provide a more oxidation-resistant ortholog (See Koths, et al., U.S. Pat. No. 4,752,585 issued Jun. 21, 1988).

### N Terminal Deletions

When produced recombinantly in bacterial expression systems directly in the absence of a leader sequence, endogenous proteases result in the deletion of the N-terminal Met-Ala1 residues to provide “desAla1” orthogonal IL2s. An orthogonal IL2 disclosed herein may comprise deletion of the first two amino acids (desAla1-desPro2) as well as substitution of the Thr3 glycosylation with a cysteine residue (T3C) to facilitate for N-terminal modification, especially PEGylation of the sulfhydryl group of the cysteine (See, e.g. Katre, et al. U.S. Pat. No. 5,206,344 issued Apr. 27, 1993).

The orthogonal IL2s may further comprise elimination of N-terminal amino acids at one or more of positions 1-9, alternatively positions 1-8, alternatively positions 1-7, alternatively positions 1-6, alternatively positions 1-5, alternatively positions 1-4, alternatively positions 1-3, alternatively positions 1-2. In some embodiments, the present disclosure provides orthogonal IL2s which are hIL2 polypeptides comprising one of the following sets of amino acid modifications:

[desAla1-desPro2-E15S-H16Q-L19V-D20L-Q22K-M23A];

[desAla1-desPro2-E15S-H16Q-L19V-D20L-Q22K];

[desAla1-desPro2-E15S-H16Q-L19V-D20L-M23A];

[desAla1-desPro2-E15S-H16Q-L19V-D20L];

[desAla1-desPro2-desThr3-E15S-H16Q-L19V-D20L-Q22K-M23A];

[desAla1-desPro2-desThr3-E15S-H16Q-L19V-D20L-Q22K];

[desAla1-desPro2-desThr3-E15S-H16Q-L19V-D20L-M23A];

[desAla1-desPro2-desThr3-E15S-H16Q-L19V-D20L];

[desAla1-desPro2-desThr3-desSer4-E15S-H16Q-L19V-D20L-Q22K-M23A];

[desAla1-desPro2-desThr3-desSer4-E15S-H16Q-L19V-D20L-Q22K];

[desAla1-desPro2-desThr3-desSer4-E15S-H16Q-L19V-D20L-M23A];

[desAla1-desPro2-desThr3-desSer4-E15S-H16Q-L19V-D20L];

[desAla1-desPro2-desThr3-desSer4-desSer5-E15S-H16Q-L19V-D20L-Q22K-M23A];

[desAla1-desPro2-desThr3-desSer4-desSer5-E15S-H16Q-L19V-D20L-Q22K];

[desAla1-desPro2-desThr3-desSer4-desSer5-E15S-H16Q-L19V-D20L-M23A];

[desAla1-desPro2-desThr3-desSer4-desSer5-E15S-H16Q-L19V-D20L];

[desAla1-desPro2-desThr3-desSer4-desSer5-desSer6-E15S-H16Q-L19V-D20L-Q22K-M23A];

[desAla1-desPro2-desThr3-desSer4-desSer5-desSer6-E15S-H16Q-L19V-D20L-Q22K];

[desAla1-desPro2-desThr3-desSer4-desSer5-desSer6-E15S-H16Q-L19V-D20L-M23A]; or

[desAla1-desPro2-desThr3-desSer4-desSer5-desSer6-E15S-H16Q-L19V-D20L].

### Minimizing Vascular Leak Syndrome

In some embodiments of the disclosure, the orthogonal IL2 comprises amino acid substitutions to avoid vascular leak syndrome, a substantial negative and dose limiting side effect of the use of IL2 therapy in human beings without out substantial loss of efficacy. See, Epstein, et al., U.S. Pat. No. 7,514,073B2 issued Apr. 7, 2009. Examples of such modifications which are included in the orthogonal IL2s of the present disclosure include one or more of R38W, R38G, R39L, R39V, F42K, and H55Y.

### Affinity Maturation:

In some embodiments, orthogonal IL2s may be affinity matured to enhance their activity with respect to the orthogonal CD122. An “affinity matured” polypeptide is one having one or more alteration(s) in one or more residues which results in an improvement in the affinity of the orthogonal polypeptide for the cognate orthogonal receptor, or vice versa, compared to a parent polypeptide which does not possess those alteration(s). Affinity maturation can be done to increase the binding affinity of the orthogonal IL2 by at least about 10%, alternatively at least about 50%, alternatively at least about 100% alternatively at least about 150%, or from 1 to 5-fold as compared to the “parent” polypeptide. An engineered orthogonal IL2 of the present invention activates its cognate orthogonal receptor, as discussed above, but has significantly reduced binding and activation of the wild-type IL2 receptor when assessed by ELISA and/or FACS analysis using sufficient amounts of the molecules under suitable assay conditions.

### Modifications to Extend Duration of Action In Vivo

As discussed above, the compositions of the present disclosure include orthogonal IL2s that have been modified to provide for an extended lifetime in vivo and/or extended duration of action in a subject. Such modifications to provided extended lifetime and/or duration of action include modifications to the primary sequence of the orthogonal IL2, conjugation to carrier molecules, (e.g. albumin, acylation, pegylation), and Fc fusions.

### Sequence Modifications to Extend Duration of Action In Vivo

As discussed above, the term orthogonal IL2 includes modifications of the orthogonal IL2 to provide for an extended lifetime in vivo and/or extended duration of action in a subject.

In some embodiments, the orthogonal IL2 may comprise certain amino acid substitutions that result in prolonged in vivo lifetime. For example, Dakshinamurthi, et al. (International Journal of Bioinformatics Research (2009) 1(2):4-13) state that one or more of the substitutions in the IL2 polypeptide V91R, K97E and T113N will result in an IL2 variant possessing enhanced stability and activity. In some embodiments, an orthogonal IL2 of the present disclosure comprises one, two or all three of the V91R, K97E and T113N modifications.

### Conjugates and Carrier Molecules

In some embodiments the orthogonal IL2 is modified to provide certain properties to the orthogonal IL2 (e.g., extended duration of action in a subject), which may be achieved through conjugation to carrier molecules to provide desired pharmacological properties such as extended half-life. In some embodiments, the orthogonal IL2 can be covalently linked to the Fc domain of IgG, albumin, or other molecules to extend its half-life, e.g., by pegylation, glycosylation, fatty acid acylation, and the like as known in the art.

### Albumin Fusions

In some embodiments, the orthogonal IL2 is expressed as a fusion protein with an albumin molecule (e.g. human serum albumin) which is known in the art to facilitate extended exposure in vivo.

In one embodiment of the invention, the hIL2 analog is conjugated to albumin, referred to herein as an “orthogonal IL2 albumin fusion.” The term “albumin” as used in the context hIL2 analog albumin fusions include albumins such as human serum albumin (HSA), cyno serum albumin, and bovine serum albumin (BSA). In some embodiments, the HSA the HSA comprises a C34S or K573P amino acid substitution relative to the wild type HSA sequence According to the present disclosure, albumin can be conjugated to an orthogonal IL2 at the carboxyl terminus, the amino terminus, both the carboxyl and amino termini, and internally (see, e.g., U.S. Pat. Nos. 5,876,969 and 7,056,701). In the HSA-orthogonal IL2 polypeptide conjugates contemplated by the present disclosure, various forms of albumin can be used, such as albumin secretion pre-sequences and variants thereof, fragments and variants thereof, and HSA variants. Such forms generally possess one or more desired albumin activities. In additional embodiments, the present disclosure involves fusion proteins comprising a hIL2 analog polypeptide fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. In some embodiments, the indirect fusion is effected by a linker such as a peptide linker or modified version thereof as more fully discussed below.

Alternatively, the hIL2 analog albumin fusion comprises orthogonal IL2s that are fusion proteins which comprise an albumin binding domain (ABD) polypeptide sequence and an orthogonal IL2 polypeptide. As alluded to above, fusion proteins which comprise an albumin binding domain (ABD) polypeptide sequence and an hIL2 analog polypeptide can, for example, be achieved by genetic manipulation, such that the nucleic acid coding for HSA, or a fragment thereof, is joined to the nucleic acid coding for the one or more orthogonal IL2 sequences. In some embodiments, the albumin-binding peptide comprises the amino acid sequence DICLPRWGCLW (SEQ ID #6).

The orthogonal IL2 polypeptide can also be conjugated to large, slowly metabolized macromolecules such as proteins; polysaccharides, such as sepharose, agarose, cellulose, or cellulose beads; polymeric amino acids such as polyglutamic acid, or polylysine; amino acid copolymers; inactivated virus particles; inactivated bacterial toxins such as toxoid from diphtheria, tetanus, cholera, or leukotoxin molecules; inactivated bacteria, dendritic cells, thyroglobulin; tetanus toxoid; Diphtheria toxoid; polyamino acids such as poly(D-lysine:D-glutamic acid); VP6 polypeptides of rotaviruses; influenza virus hemaglutinin, influenza virus nucleoprotein; Keyhole Limpet Hemocyanin (KLH); and hepatitis B virus core protein and surface antigen Such conjugated forms, if desired, can be used to produce antibodies against a polypeptide of the present disclosure.

In some embodiments, the orthogonal IL2 is conjugated (either chemically or as a fusion protein) with an XTEN which provides extended duration of akin to pegylation and may be produced as a recombinant fusion protein in E. coli. XTEN polymers suitable for use in conjunction with the orthogonal IL2s of the present disclosure are provided in Podust, et al. (2016) “Extension of in vivo half-life of biologically active molecules by XTEN protein polymers”, J Controlled Release 240:52-66 and Haeckel et al. (2016) “XTEN as Biological Alternative to PEGylation Allows Complete Expression of a Protease-Activatable Killin-Based Cytostatic” PLOS ONE DOI:10.1371/journal.pone.0157193 Jun. 13, 2016. The XTEN polymer fusion protein may incorporate a protease sensitive cleavage site between the XTEN polypeptide and the orthogonal IL2 such as an MMP-2 cleavage site.

Additional candidate components and molecules for conjugation include those suitable for isolation or purification. Particular non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes.

In some embodiments, the IL-2 mutein also may be linked to additional therapeutic agents including therapeutic compounds such as anti-inflammatory compounds or antineoplastic agents, therapeutic antibodies (e.g. Herceptin), immune checkpoint modulators, immune checkpoint inhibitors (e.g. anti-PD1 antibodies), cancer vaccines as described elsewhere in this disclosure. Anti-microbial agents include aminoglycosides including gentamicin, antiviral compounds such as rifampicin, 3′-azido-3′-deoxythymidine (AZT) and acylovir, antifungal agents such as azoles including fluconazole, plyre macrolides such as amphotericin B, and candicidin, anti-parasitic compounds such as antimonials, and the like. The orthogonal IL2 may be conjugated to additional cytokines as CSF, GSF, GMCSF, TNF, erythropoietin, immunomodulators or cytokines such as the interferons or interleukins, a neuropeptide, reproductive hormones such as HGH, FSH, or LH, thyroid hormone, neurotransmitters such as acetylcholine, hormone receptors such as the estrogen receptor. Also included are non-steroidal anti-inflammatories such as indomethacin, salicylic acid acetate, ibuprofen, sulindac, piroxicam, and naproxen, and anesthetics or analgesics. Also included are radioisotopes such as those useful for imaging as well as for therapy.

The orthogonal IL2s of the present disclosure may be chemically conjugated to such carrier molecules using well known chemical conjugation methods. Bi-functional cross-linking reagents such as homofunctional and heterofunctional cross-linking reagents well known in the art can be used for this purpose. The type of cross-linking reagent to use depends on the nature of the molecule to be coupled to IL-2 mutein and can readily be identified by those skilled in the art. Alternatively, or in addition, the orthogonal IL2 and/or the molecule to which it is intended to be conjugated may be chemically derivatized such that the two can be conjugated in a separate reaction as is also well known in the art.

### Pegylation:

In some embodiments, the orthogonal IL2 is conjugated to one or more water-soluble polymers. Examples of water soluble polymers useful in the practice of the present invention include polyethylene glycol (PEG), poly-propylene glycol (PPG), polysaccharides (polyvinylpyrrolidone, copolymers of ethylene glycol and propylene glycol, poly(oxyethylated polyol), polyolefinic alcohol, polysaccharides, poly-alpha-hydroxy acid, polyvinyl alcohol (PVA), polyphosphazene, polyoxazolines (POZ), poly(N-acryloylmorpholine), or a combination thereof.

In some embodiments the orthogonal IL2 is conjugated to one or more polyethylene glycol molecules or “pegylated.” Although the method or site of PEG attachment to orthogonal IL2 may vary, in certain embodiments the pegylation does not alter, or only minimally alters, the activity of the orthogonal IL2.

In some embodiments, a cysteine may be substituted for the threonine at position 3 (3TC) to facilitate N-terminal pegylation using particular chemistries.

In some embodiments, selective pegylation of the orthogonal IL2 (for example by the incorporation of non-natural amino acids having side chains to facilitate selective PEG conjugation chemistries as described Ptacin, et al., (PCT International Application No. PCT/US2018/045257 filed Aug. 3, 2018 and published Feb. 7, 2019 as International Publication Number WO 2019/028419A1 may be employed to generate an orthogonal IL2 with having reduced affinity for one or more subunits (e.g. CD25, CD132) of an IL2 receptor complex. For example, an orthogonal IL2 incorporating non-natural amino acids having a PEGylatable specific moiety at those sequences or residues of IL2 identified as interacting with CD25 including amino acids 34-45, 61-72 and 105-109 typically provides an orthogonal IL2 having diminished binding to CD25. Similarly, an orthogonal IL2 incorporating non-natural amino acids having a PEGylatable specific moiety at those sequences or residues of IL2 identified as interacting with hCD132 including amino acids 18, 22, 109, 126, or from 119-133 provides an orthogonal IL2 having diminished binding to hCD132.

In certain embodiments, the increase in half-life is greater than any decrease in biological activity. PEGs suitable for conjugation to a polypeptide sequence are generally soluble in water at room temperature, and have the general formula R(O—CH2-CH2)nO-R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure.

A molecular weight of the PEG used in the present disclosure is not restricted to any particular range. The PEG component of the PEG-orthogonal IL2 can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa or from about 10 kDa to about 30 kDa. Linear or branched PEG molecules having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, alternatively about 30,000 to about 40,000 daltons. In one embodiment of the invention, the PEG is a 40 kD branched PEG comprising two 20 kD arms.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. Chromatography may be used to resolve conjugate fractions, and a fraction is then identified which contains the conjugate having, for example, the desired number of PEGs attached, purified free from unmodified protein sequences and from conjugates having other numbers of PEGs attached.

PEGs suitable for conjugation to a polypeptide sequence are generally soluble in water at room temperature, and have the general formula R(O-CH2-CH2)nO-R, where R is hydrogen or a protective group such as an alkyl or an alkanol group, and where n is an integer from 1 to 1000. When R is a protective group, it generally has from 1 to 8 carbons.

Two widely used first generation activated monomethoxy PEGs (mPEGs) are succinimdyl carbonate PEG (SC-PEG; see, e.g., Zalipsky, et al. (1992) Biotehnol. Appl. Biochem 15:100-114) and benzotriazole carbonate PEG (BTC-PEG; see, e.g., Dolence, et al. U.S. Pat. No. 5,650,234), which react preferentially with lysine residues to form a carbamate linkage but are also known to react with histidine and tyrosine residues. Use of a PEG-aldehyde linker targets a single site on the N-terminus of a polypeptide through reductive amination.

Pegylation most frequently occurs at the □-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General pegylation strategies known in the art can be applied herein.

The PEG can be bound to an orthogonal IL2 of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide.

In some embodiments, the pegylation of orthogonal IL2s is facilitated by the incorporation of non-natural amino acids bearing unique side chains to facilitate site specific pegylation. The incorporation of non-natural amino acids into polypeptides to provide functional moieties to achieve site specific pegylation of such polypeptides is known in the art. See e.g. Ptacin, et al., (PCT International Application No. PCT/US2018/045257 filed Aug. 3, 2018 and published Feb. 7, 2019 as International Publication Number WO 2019/028419A1. In one embodiment, the orthogonal IL2s of the present invention incorporate a non-natural amino acid at position D109 of the orthogonal IL2. In one embodiment of the invention the orthogonal IL2 is a PEGylated at position 109 of the orthogonal IL2 to a PEG molecule having a molecular weight of about 20 kD, alternatively about 30 kD, alternatively about 40 kD.

The PEG conjugated to the polypeptide sequence can be linear or branched. Branched PEG derivatives, “star-PEGs” and multi-armed PEGs are contemplated by the present disclosure. Specific embodiments PEGs useful in the practice of the present invention include a 10 kDa linear PEG-aldehyde (e.g., Sunbright® ME-100AL, NOF America Corporation, One North Broadway, White Plains, N.Y. 10601 USA), 10 kDa linear PEG-NHS ester (e.g., Sunbright® ME-100CS, Sunbright® ME-100AS, Sunbright® ME-100GS, Sunbright® ME-100HS, NOF), a 20 kDa linear PEG-aldehyde (e.g. Sunbright® ME-200AL, NOF, a 20 kDa linear PEG-NHS ester (e.g., Sunbright® ME-200CS, Sunbright® ME-200AS, Sunbright® ME-200GS, Sunbright® ME-200HS, NOF), a 20 kDa 2-arm branched PEG-aldehyde the 20 kDA PEG-aldehyde comprising two 10 kDA linear PEG molecules (e.g., Sunbright® GL2-200AL3, NOF), a 20 kDa 2-arm branched PEG-NHS ester the 20 kDA PEG-NHS ester comprising two 10 kDA linear PEG molecules (e.g., Sunbright® GL2-200TS, Sunbright® GL200GS2, NOF), a 40 kDa 2-arm branched PEG-aldehyde the 40 kDA PEG-aldehyde comprising two 20 kDA linear PEG molecules (e.g., Sunbright® GL2-400AL3), a 40 kDa 2-arm branched PEG-NHS ester the 40 kDA PEG-NHS ester comprising two 20 kDA linear PEG molecules (e.g., Sunbright® GL2-400AL3, Sunbright® GL2-400GS2, NOF), a linear 30 kDa PEG-aldehyde (e.g., Sunbright® ME-300AL) and a linear 30 kDa PEG-NHS ester.

As previously noted, the PEG may be attached directly to the orthogonal IL2 or via a linker molecule. Suitable linkers include “flexible linkers” which are generally of sufficient length to permit some movement between the modified polypeptide sequences and the linked components and molecules. The linker molecules are generally about 6-50 atoms long. The linker molecules can also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Examples of flexible linkers include glycine polymers (G)n, glycine-serine polymers, glycine-alanine polymers, alanine-serine polymers, and other flexible linkers. Glycine and glycine-serine polymers are relatively unstructured, and therefore can serve as a neutral tether between components. Further examples of flexible linkers include glycine polymers (G)n, glycine-alanine polymers, alanine-serine polymers, glycine-serine polymers. Glycine and glycine-serine polymers are relatively unstructured, and therefore may serve as a neutral tether between components. A multimer (e.g., 1, 2, 3, 4, 5, 6, 7, 8, 9, 10, 10-20, 20-30, or 30-50) of these linker sequences may be linked together to provide flexible linkers that may be used to conjugate a heterologous amino acid sequence to the polypeptides disclosed herein.

Further, such linkers may be used to link the orthogonal IL2 to additional heterologous polypeptide components as described herein, the heterologous amino acid sequence may be a signal sequence and/or a fusion partner, such as, albumin, Fc sequence, and the like.

In one embodiment of the disclosure, the orthogonal IL2 is a human orthogonal IL2 of the formula 2:
40 kD-branched-PEG-linker-desAla1-hIL2[E15S-H16Q-L19V-D20L-Q22K-M23A]-COOH.

In another embodiment of the invention, the orthogonal IL2 is a human orthogonal IL2 of the formula 3:

(SEQ ID NO: 23)
40KdPEG-(linker)n-PTSSSTKKTQLQLSQLLVLLKAILNGINNYK
NPKLTRMLTFKFYMPKKATELKHLQCLEEELKPLEEVLNLAQSKNFHLR
PRDLISNINVIVLELKGSETTFMCEYADETATIVEFLNRWITFCQSIIS
TLT
wherein n = 0 or 1.

### Acylation

In some embodiments, the orthogonal IL2 of the present disclosure may be acylated by conjugation to a fatty acid molecule as described in Resh (2016) Progress in Lipid Research 63: 120-131. Examples of fatty acids that may be conjugated include myristate, palmitate and palmitoleic acid. Myristoylate is typically linked to an N-terminal glycine but lysines may also be myristoylated. Palmitoylation is typically achieved by enzymatic modification of free cysteine —SH groups such as DHHC proteins catalyze S-palmitoylation. Palmitoleylation of serine and threonine residues is typically achieved enzymatically using PORCN enzymes.

### Acetylation

In some embodiments, the IL-2 mutein is acetylated at the N-terminus by enzymatic reaction with N-terminal acetyltransferase and, for example, acetyl CoA. Alternatively, or in addition to N-terminal acetylation, the IL-2 mutein is acetylated at one or more lysine residues, e.g. by enzymatic reaction with a lysine acetyltransferase. See, for example Choudhary et al. (2009) Science 325 (5942):834L2 ortho840.

### Fc Fusions

In some embodiments, the IL2 fusion protein may incorporate an Fc region derived from the IgG subclass of antibodies that lacks the IgG heavy chain variable region. The “Fc region” can be a naturally occurring or synthetic polypeptide that is homologous to the IgG C-terminal domain produced by digestion of IgG with papain. IgG Fc has a molecular weight of approximately 50 kDa. The mutant IL-2 polypeptides can include the entire Fc region, or a smaller portion that retains the ability to extend the circulating half-life of a chimeric polypeptide of which it is a part. In addition, full-length or fragmented Fc regions can be variants of the wild type molecule. That is, they can contain mutations that may or may not affect the function of the polypeptides; as described further below, native activity is not necessary or desired in all cases. In certain embodiments, the IL-2 mutein fusion protein (e.g., an IL-2 partial agonist or antagonist as described herein) includes an IgG1, IgG2, IgG3, or IgG4 Fc region. Exemplary Fc regions can include a mutation that inhibits complement fixation and Fc receptor binding, or it may be lytic, i.e., able to bind complement or to lyse cells via another mechanism such as antibody-dependent complement lysis (ADCC).

In some embodiments, the orthogonal IL2 comprises a functional domain of an Fc-fusion chimeric polypeptide molecule. Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates. The “Fc region” useful in the preparation of Fc fusions can be a naturally occurring or synthetic polypeptide that is homologous to an IgG C-terminal domain produced by digestion of IgG with papain. IgG Fc has a molecular weight of approximately 50 kDa. The orthogonal IL2s may provide the entire Fc region, or a smaller portion that retains the ability to extend the circulating half-life of a chimeric polypeptide of which it is a part. In addition, full-length or fragmented Fc regions can be variants of the wild type molecule. In a typical presentation, each monomer of the dimeric Fc carries a heterologous polypeptide, the heterologous polypeptides being the same or different.

In some embodiments, when the orthogonal IL2 is to be administered in the format of an Fc fusion, particularly in those situations when the polypeptide chains conjugated to each subunit of the Fc dimer are different, the Fc fusion may be engineered to possess a “knob-into-hole modification.” The knob-into-hole modification is more fully described in Ridgway, et al. (1996) Protein Engineering 9(7):617-621 and U.S. Pat. No. 5,731,168, issued Mar. 24, 1998. The knob-into-hole modification refers to a modification at the interface between two immunoglobulin heavy chains in the CH3 domain, wherein: i) in a CH3 domain of a first heavy chain, an amino acid residue is replaced with an amino acid residue having a larger side chain (e.g. tyrosine or tryptophan) creating a projection from the surface (“knob”) and ii) in the CH3 domain of a second heavy chain, an amino acid residue is replaced with an amino acid residue having a smaller side chain (e.g. alanine or threonine), thereby generating a cavity (“hole”) within at interface in the second CH3 domain within which the protruding side chain of the first CH3 domain (“knob”) is received by the cavity in the second CH3 domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. Furthermore, the Fc domains may be modified by the introduction of cysteine residues at positions 5354 and Y349 which results in a stabilizing disulfide bridge between the two antibody heavy chains in the Fe region (Carter, et al. (2001) Immunol Methods 248, 7-15). The knob-into-hole format is used to facilitate the expression of a first polypeptide (e.g. an orthogonal IL2) on a first Fc monomer with a “knob” modification and a second polypeptide on the second Fc monomer possessing a “hole” modification to facilitate the expression of heterodimeric polypeptide conjugates.

The Fc region can be “lytic” or “non-lytic,” but is typically non-lytic. A non-lytic Fc region typically lacks a high affinity Fc receptor binding site and a C1q binding site. The high affinity Fc receptor binding site of murine IgG Fc includes the Leu residue at position 235 of IgG Fc. Thus, the Fc receptor binding site can be inhibited by mutating or deleting Leu 235. For example, substitution of Glu for Leu 235 inhibits the ability of the Fc region to bind the high affinity Fc receptor. The murine C1q binding site can be functionally destroyed by mutating or deleting the Glu 318, Lys 320, and Lys 322 residues of IgG. For example, substitution of Ala residues for Glu 318, Lys 320, and Lys 322 renders IgG1 Fc unable to direct antibody-dependent complement lysis. In contrast, a lytic IgG Fc region has a high affinity Fc receptor binding site and a C1q binding site. The high affinity Fc receptor binding site includes the Leu residue at position 235 of IgG Fc, and the C1q binding site includes the Glu 318, Lys 320, and Lys 322 residues of IgG 1. Lytic IgG Fc has wild type residues or conservative amino acid substitutions at these sites. Lytic IgG Fc can target cells for antibody dependent cellular cytotoxicity or complement directed cytolysis (CDC). Appropriate mutations for human IgG are also known (see, e.g., Morrison et al., The Immunologist 2:119-124, 1994; and Brekke et al., The Immunologist 2: 125, 1994).

In certain embodiments, the amino- or carboxyl-terminus of an orthogonal IL2 of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates.

In some embodiments, the Fc domain monomer comprises at least one mutation relative to a wild-type human IgG1, IgG2, or IgG4 Fc region as described in United States Patent No. U.S. Ser. No. 10/259,859B2, the entire teaching of which is herein incorporated by reference. As disclosed therein, the Fc domain monomer comprises:
(a) one of the following amino acid substitutions relative to wild type human IgG1:
   T366W, T366S, L368A, Y407V, T366Y, T394W, F405W, Y349T, Y349E, Y349V, L351T, L351H, L351N, L351K, P353S, S354D, D356K, D356R, D356S, E357K, E357R, E357Q, S364A, T366E, L368T, L368Y, L368E, K370E, K370D, K370Q, K392E, K392D, T394N, P395N, P396T, V397T, V397Q, L398T, D399K, D399R, D399N, F405T, F405H, F405R, Y407T, Y407H, Y4071, K409E, K409D, K409T, or K4091; or
(b) (i) a N297A mutation relative to a human IgG1 Fc region;
   (ii) a L234A, L235A, and G237A mutation relative to a human IgG1 Fc region;
   (iii) a L234A, L235A, G237A, and N297A mutation relative to a human IgG1 Fc region;
   (iv) a N297A mutation relative to a human IgG2 Fc region;
   (v) a A330S and P331S mutation relative to a human IgG2 Fc region;
   (vi) a A330S, P331S, and N297A mutation relative to a human IgG2 Fc region;
   (vii) a S228P, E233P, F234V, L235A, and delG236 mutation relative to a human IgG4 Fc region; or
   (viii) a S228P, E233P, F234V, L235A, delG236, and N297A mutation relative to a human IgG4 Fc region.

In some embodiments, the Fc domain monomer comprises:
(a) one of the following amino acid substitutions relative to wild type human IgG1: T366W, T366S, L368A, Y407V, T366Y, T394W, F405W, Y349T, Y349E, Y349V, L35 IT, L351H, L351N, L351K, P353S, S354D, D356K, D356R, D356S, E357K, E357R, E357Q, S364A, T366E, L368T, L368Y, L368E, K370E, K370D, K370Q. K392E, K392D, T394N, P395N, P396T, V397T, V397Q, L398T, D399K, D399R, D399N, F405T, F405H, F405R, Y407T, Y407H, Y4071, K409E, K409D, K409T, or K4091; and
(b) the Fc domain monomer further comprises
   (i) a N297A mutation relative to a human IgG1 Fc region;
   (ii) a L234A, L235A, and G237A mutation relative to a human IgG1 Fc region;
   (iii) a L234A, L235A, G237A, and N297A mutation relative to a human IgG1 Fc region;
   (iv) a N297A mutation relative to a human IgG2 Fc region;
   (v) a A330S and P331S mutation relative to a human IgG2 Fc region;
   (vi) a A330S, P331S, and N297A mutation relative to a human IgG2 Fc region;
   (vii) a S228P, E233P, F234V, L235A, and delG236 mutation relative to a human IgG4 Fc region; or
   (viii) a S228P, E233P, F234V, L235A, delG236, and N297A mutation relative to a human IgG4 Fc region.

In some embodiments, the polypeptide exhibits a reduction of phagocytosis in a phagocytosis assay compared to a polypeptide with a wild-type human IgG Fc region. In some embodiments, the Fc domain monomer is linked to a second polypeptide comprising a second Fc domain monomer to form an Fc domain dimer.

### Chimeric Polypeptides/Fusion Proteins

In some embodiments, embodiment, the orthogonal IL2 may comprise a functional domain of a chimeric polypeptide. orthogonal IL2 fusion proteins of the present disclosure may be readily produced by recombinant DNA methodology by techniques known in the art by constructing a recombinant vector comprising a nucleic acid sequence comprising a nucleic acid sequence encoding the orthogonal IL2 in frame with a nucleic acid sequence encoding the fusion partner either at the N-terminus or C-terminus of the orthogonal IL2, the sequence optionally further comprising a nucleic acid sequence in frame encoding a linker or spacer polypeptide.

### Flag Tags

In other embodiments, the orthogonal IL2 can be modified to include an additional polypeptide sequence that functions as an antigenic tag, such as a FLAG sequence. FLAG sequences are recognized by biotinylated, highly specific, anti-FLAG antibodies, as described herein (see e.g., Blanar et al. (1992) Science 256:1014 and LeClair, et al. (1992) PNAS-USA 89:8145). In some embodiments, the orthogonal IL2 polypeptide further comprises a C-terminal c-myc epitope tag.

### His Tags

In some embodiment, the orthogonal IL2s (including fusion proteins of such orthogonal IL2s) of the present invention are expressed as a fusion protein with one or more transition metal chelating polypeptide sequences. The incorporation of such a transition metal chelating domain facilitates purification immobilized metal affinity chromatography (IMAC) as described in Smith, et al. U.S. Pat. No. 4,569,794 issued Feb. 11, 1986. Examples of transition metal chelating polypeptides useful in the practice of the present invention are described in Smith, et al. supra and Dobeli, et al. U.S. Pat. No. 5,320,663 issued May 10, 1995, the entire teachings of which are hereby incorporated by reference. Particular transition metal chelating polypeptides useful in the practice of the present invention are peptides comprising 3-6 contiguous histidine residues such as a six-histidine peptide (His)6 and are frequently referred to in the art as “His-tags.”

### Targeted Orthogonal IL2 Fusion Proteins:

In some embodiments, the orthogonal IL2 is provided as a fusion protein with a polypeptide sequence (“targeting domain”) to facilitate selective binding to particular cell type or tissue expressing a cell surface molecule that specifically binds to such targeting domain, optionally incorporating a linker molecule of from 1-40 (alternatively 2-20, alternatively 5-20, alternatively 10-20) amino acids between the orthogonal IL2 sequence and the sequence of the targeting domain of the fusion protein.

In other embodiments, a chimeric polypeptide including a mutant IL-2 and an antibody or antigen-binding portion thereof can be generated. The antibody or antigen-binding component of the chimeric protein can serve as a targeting moiety. For example, it can be used to localize the chimeric protein to a particular subset of cells or target molecule. Methods of generating cytokine-antibody chimeric polypeptides are described, for example, in U.S. Pat. No. 6,617,135.

In some embodiments, the targeting domain of the orthogonal IL2 fusion protein specifically binds to a cell surface molecule of a tumor cell. In one embodiment wherein the ECD of the CAR of a CAR-T cell specifically binds to CD-19, the orthogonal IL2 may be provided as a fusion protein with a CD-19 targeting moiety. For example, in one embodiment wherein the ECD of the CAR of an CAR-T cell is an scFv molecule that provides specific binding to CD-19, the orthogonal IL2 is provided as a fusion protein with a CD-19 targeting moiety such as a single chain antibody (e.g., an scFv or VHH) that specifically binds to CD-19.

In some embodiments, the fusion protein comprises an IL-2 mutein and the anti-CD19 sdFv FMC63 (Nicholson, et al. (1997) Mol Immunol 34: 1157-1165). Similarly, in some embodiments wherein the ECD of the CAR of an CAR-T cell specifically binds to BCMA, the orthogonal IL2 is provided as a fusion protein with a BCMA targeting moiety, such as antibody comprising the CDRs of anti-BMCA antibodies as described in in Kalled, et al. (U.S. Pat. No. 9,034,324 issued May 9, 2015) or antibodies comprising the CDRs as described in Brogdon, et al., (U.S. Pat. No. 10,174,095 issued Jan. 8, 2019). In some embodiments the orthogonal IL2 is provided as a fusion protein with a GD2 targeting moiety, such as an antibody comprising the CDRs of described in Cheung, et al., (U.S. Pat. No. 9,315,585 issued Apr. 19, 2016) or the CDRs derived from ME36.1 (Thurin et al., (1987) Cancer Research 47:1229-1233), 14G2a, 3F8 (Cheung, et al., 1985 Cancer Research 45:2642-2649), hu14.18, 8B6, 2E12, or ic9.

In an alternative embodiment, the targeted orthogonal IL2s of the present disclosure may be administered in combination with CAR-T cell therapy to provide targeted delivery of the orthogonal IL2 to the CAR-T cell based on an extracellular receptor of the CAR-T cell such as by and anti-FMC63 antibody to target the IL2 activity to the CAR-T cells and rejuvenate exhausted CAR-T cells in vivo. Consequently, embodiments of the present disclosure include targeted delivery of orthogonal IL2s by conjugation of such orthogonal IL2s to antibodies or ligands that are designed to interact with specific cell surface molecules of CAR-T cells. An example of such a molecule would an anti-FMC63-orthogonal IL2.

In other embodiments, the chimeric polypeptide includes the mutant IL-2 polypeptide and a heterologous polypeptide that functions to enhance expression or direct cellular localization of the mutant IL-2 polypeptide, such as the Aga2p agglutinin subunit (see, e.g., Boder and Wittrup, Nature Biotechnol. 15:553-7, 1997).

### Protein Transduction Domain Fusion Proteins

In some embodiments, the orthogonal IL2 further comprises a “Protein Transduction Domain” or “PTD.” A PTD is a polypeptide, polynucleotide, carbohydrate, or organic or inorganic molecule that facilitates traversing a lipid bilayer, micelle, cell membrane, organelle membrane, or vesicle membrane. The incorporation of a PTD into an orthogonal IL2 facilitates the molecule traversing a membrane. In some embodiments, a PTD is covalently linked to the amino or carboxy terminus of an orthogonal IL2. In some embodiments, the PTD is incorporated as part of an PTD-orthogonal IL2 fusion protein, either at the N or C terminus of the molecule.

Exemplary protein transduction domains include, but are not limited to, a minimal decapeptide protein transduction domain (corresponding to residues 47-57 of HIV-1 TAT); a polyarginine sequence comprising a number of arginine residues sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008), Transportan (as described in Wierzbicki, et al., (2014) Folio Histomchemica et Cytobiologica 52(4): 270-280 and Pooga, et a (1998) FASEB J 12(1)67-77 and commercially available from AnaSpec as Catalog No. AS-61256); KALA (as decribed in Wyman et al., (1997) Biochemistry 36(10) 3008-3017 and commercially available from AnaSpec as Catalog No. AS-65459); Antennapedia Peptide (as described in Pietersz et al., (2001) Vaccine 19:1397 and commercially available from AnaSpec as Catalog No. AS-61032); TAT 47-57 (commercially available from AnaSpec as Catalog No. AS-60023).

In some embodiments, the IL-2 conjugate comprises a plasma half-life in a human subject of greater than 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 10 days, 14 days, or 30 days.

### Orthogonal IL2 Specific Configurations of Modified CD122

The STAT3 binding motifs can be at the C-terminus of the intracellular domain or as an internal sequence of a human CD122 (e.g., a human orthogonal CD122 or a native human CD122).

Modified CD122 Embodiments that Comprise One or More STAT3 Binding Motifs Fused to the C-Terminus of a Human CD122

In some cases, the modified human CD122 comprises one or more STAT3 binding motifs fused to the C-terminus of the intracellular domain of a human CD122. The human CD122 can be a native human CD122 (SEQ ID NO: 1) or a orthogonal human CD122 as disclosed above.

TABLE 1
Exemplary fusion proteins, below:
Human orthogonal CD 122 chimeras

Ortho-CD 122-GGYLRQ
Ortho-CD 122-GGYLKQ
Ortho-CD 122-GGYRHQ
Ortho-CD 122-GGYLRQ
Ortho-CD 122-GGYFKQ
Ortho-CD 122-GGYLPQ
Ortho-CD 122-GGYMPQ
Ortho-CD 122-GGYDKPH

The term “Ortho-CD122” refers to any of the orthogonal human CD122 proteins disclosed herein.

In some cases, the modified human CD122 comprises a STAT3 binding motif that is connected to the human CD122 through a linker. Linkers can be derived from naturally-occurring proteins or synthesis sequences. Methods for designing linkers are well-known in the art, for example, as disclosed in Chen et al. Adv. Drug. Deliv. Rev. 2013 Oct. 15; 65(10): 1357-1369, the relevant portion thereof is herein incorporated by reference. In some cases, the linker consists of 1-20 amino acid residues, 1-10 amino acid residues, or 1-5 amino acid residues. In some embodiments, the linker comprises a dinucleotide Gn, where n can be 1-10, or from 1-5, e.g., from 2-4.

The modified human CD122 may comprise one or more STAT3 binding motifs and one or more linker sequences. Said linker sequences may connect the human CD122 and one of the STAT3 binding motifs or connect individual STAT3 binding motifs. The one or more linker sequence may have the same or different sequences.

### Modified CD122 Embodiments Comprising a STAT3 Recognition Motif as an the Internal Sequence of the Intracellular Domain of CD122.

In some embodiments, one or more STAT3 binding motifs are present as an internal (i.e., at neither C nor N terminus) sequence of CD122. A modified human CD122 of this configuration can be produced by identifying a suitable region within the native human CD122 or human orthogonal CD122 coding sequence that can be mutated to encode a STAT3 binding motif. In one embodiment, the region has a sequence similar to the STAT3 binding motif, for example a region comprising a four-nucleotide sequence that begins with a tyrosine residue. One such region in the native human CD122 encodes a sequence of YFTYDPYSEE, which is located between position 355 and position 364 of the native human CD122 protein. In some embodiments, one or two of the YFTY, YDPY, or YSEE comprised in this region are substituted with a STAT3 recognition motif to produce a modified human CD122 disclosed herein.

The modified human CD122 are able to induce STAT3 and STAT5 signaling upon binding to a cognate IL2 ligand and the ability can be confirmed by e.g., detecting phorphorylation of STAT3 and STAT5. For example, the modified human CD122 can be introduced and expressed in T cells and antibodies that are specific to phospho-STAT5 and phosphor-STAT3 are used to detect the phosphorylation of STAT3 and STAT5. One exemplary method for detecting a recombinant protein's ability to induce STAT3 and STAT5 signaling is described in Kagoya et al. Nat Med. 2018 March; 24(3): 352-359. doi:10.1038/nm.4478, at p7.

### Polynucleotides and Expression Vectors

Polynucleotides encoding the modified human CD122 can be produced using techniques well known in the art. In some cases, the modified human CD122 may be produced by any conventional method including recombinant or solid phase synthesis. In some embodiments, the modified human CD122 is produced by recombinant methods. A nucleic acid sequence encoding the be introduced on an expression vector into the cell to be engineered. DNA encoding a modified human orthogonal CD122 may be obtained from various sources as designed during the engineering process. Amino acid sequence variants of the native human CD122 polypeptides to the produce the modified human CD122 of the present disclosure are prepared by introducing appropriate nucleotide changes into the coding sequence, as described herein. Such variants represent insertions, substitutions, and/or specified deletions of, residues as noted. Any combination of insertion, substitution, and/or specified deletion is made to arrive at the final construct, provided that the final construct possesses the desired biological activity as defined herein.

To express the modified CD122, a nucleic acid encoding the modified CD122 is inserted into a replicable vector for expression. Many such vectors are available. The vector components generally include, but are not limited to, one or more of the following: an origin of replication, one or more marker genes, an enhancer element, a promoter, and a transcription termination sequence. Vectors include viral vectors, plasmid vectors, integrating vectors, and the like. Plasmids are examples of non-viral vectors. In order to facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, and magnetic fields (electroporation).

A modified CD122 may be produced recombinantly not only directly, but also as a fusion polypeptide with a heterologous polypeptide, e.g. a signal sequence or other polypeptide having a specific cleavage site at the N-terminus of the mature protein or polypeptide. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. In mammalian cell expression the native signal sequence may be used, or other mammalian signal sequences may be suitable, such as signal sequences from secreted polypeptides of the same or related species, as well as viral secretory leaders, for example, the herpes simplex gD signal.

Also provided are expression vectors that can be used to introduce the polynucleotides encoding the modified CD122 into the cell. Various vectors are known in the art and can be used for this purpose, e.g., viral vectors, plasmid vectors, minicircle vectors. Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors will contain a promoter that is recognized by the host organism and is operably linked to an orthogonal protein coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Transcription from vectors in mammalian host cells may be controlled, for example, by promoters obtained from the genomes of viruses such as polyoma virus, fowlpox virus, adenovirus (such as human adenovirus serotype 5), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence but is preferably located at a site 5′ from the promoter. Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

Suitable host cells for cloning or expressing the coding sequence for the modified CD122 are the prokaryote, yeast, or higher eukaryote cells described above. In some embodiments, host cells are human immune cells, e.g., T cells. In some embodiments, the host cells are CAR-T cells, as further described below.

In some embodiments, the host cells are mammalian host cell lines, for example, mouse L cells (L-M[TK-], ATCC #CRL-2648), monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); Chinese hamster ovary cells/-DHFR (CHO); mouse sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

Host cells comprising the coding sequence of the modified CD122 may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression and will be apparent to the ordinarily skilled artisan.

In some embodiments, host cells, such as T cells, are transformed with an expression vector comprising a nucleic acid sequence encoding the modified CD122. A IL2 ligand (e.g., an orthogonal IL2) may be employed in methods of selectively expanding such engineered T cells (e.g., human T-cells) which have been engineered to express a corresponding modified human CD122 (e.g., a modified orthogonal CD122). T-cells useful for engineering with the constructs described herein include naïve T-cells, central memory T-cells, effector memory T-cells or combination thereof. T cells for engineering as described above are collected from a subject or a donor may be separated from a mixture of cells by techniques that enrich for desired cells or may be engineered and cultured without separation. Alternatively, the T cells for engineering may be separated from other cells. Techniques providing accurate separation include fluorescence activated cell sorters. The cells may be selected against dead cells by employing dyes associated with dead cells (e.g., propidium iodide). The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS). The collected and optionally enriched cell population may be used immediately for genetic modification or may be frozen at liquid nitrogen temperatures and stored, being thawed and capable of being reused. The cells will usually be stored in 10% DMSO, 50% FCS, 40% RPMI 1640 medium.

Engineered Immune Cells that Express the Modified CD122

Also provided herein are immune cells that express a modified human CD122. Immune cells can be, for example, T cells (e.g., CD4+ cells, Cd8+ cells) and NK cells. In some embodiments the T cell includes without limitation naïve CD8+ T cells, cytotoxic CD8+ T cells, naïve CD4+ T cells, helper T cells, e.g. TH1, TH2, TH9, TH11, TH22, TFH; regulatory T cells, e.g. TR1, Tregs, inducible Tregs; memory T cells, e.g. central memory T cells, effector memory T cells, NKT cells, tumor infiltrating lymphocytes (TILs) and engineered variants of such T-cells including but not limited to CAR-T cells, recombinantly modified TILs and TCR engineered cells. In some embodiments, the engineered cells comprise a complex mixture of immune cells, e.g., tumor infiltrating lymphocytes (TILs) isolated from an individual in need of treatment. See, for example, Yang and Rosenberg (2016) Adv Immunol. 130:279-94, “Adoptive T Cell Therapy for Cancer; Feldman et al (2015) Seminars in Oncol. 42(4):626-39 “Adoptive Cell Therapy-Tumor-Infiltrating Lymphocytes, T-Cell Receptors, and Chimeric Antigen Receptors”; Clinical Trial NCT01174121, “Immunotherapy Using Tumor Infiltrating Lymphocytes for Patients With Metastatic Cancer”; Tran et al. (2014) Science 344(6184)641-645, “Cancer immunotherapy based on mutation-specific CD4+ T cells in a patient with epithelial cancer”.

### CAR-T Cells

In some embodiments the engineered immune cells also express a chimeric antigen receptor (CAR). CARs allows the immune cells able to bind to the antigen on the diseased cells, for example, tumor cells, and thus enable specific killing of the diseased cells. The antigen binding domain (ABD) of the of the CAR may be monovalent or multivalent and comprise one or multiple (e.g. 1, 2, or 3) polypeptide sequences (e.g. scFv, VHH, and/ligands ligand) that specifically bind to a cell surface tumor antigen. In some embodiments, tumor antigens and CARs comprising ABDs that selectively bind to such cell surface tumor are known in the art (see, e.g., Dotti, et al., Immunol Rev. 2014 January; 257(1). The methods and compositions of the present disclosure are useful in conjunction with CAR therapy wherein the ABD of the CAR specifically binds a tumor antigen including but not limited to CD123, CD19, CD20, BCMA, CD22, CD30, CD70, Lewis Y, GD3, GD3, mesothelin, ROR CD44, CD171, EGP2, EphA2, ErbB2, ErbB3/4, FAP, FAR IL11Ra, PSCA, PSMA and NCAM. Antibodies reactive with these targets are well known in the literature and one of skill in the art is capable of isolating the CDRs from such antibodies for the construction of polypeptide sequences of single chain antibodies (e.g. scFvs, CDR grafted VHHs and the like) that may be incorporated into the ABD of the CAR.

In one embodiment of the invention the engineered immune cell is a T-cell expressing the modified CD122 and also expressing a chimeric antigen receptor. The cell is known as a ‘CAR-T’ cell.

A CAR typically comprise an antigen binding domain (ABD), which can specifically binds to an antigen expressed on the surface of a target cell. The ABD may be any polypeptide that specifically binds to one or more antigens expressed on the surface of a target cell. A CAR can further comprise a transmembrane domain joining the ABD (or linker, if employed) to the intracellular cytoplasmic domain of the CAR. The transmembrane domain is comprised of any polypeptide sequence which is thermodynamically stable in a eukaryotic cell membrane. The transmembrane spanning domain may be derived from the transmembrane domain of a naturally occurring membrane spanning protein or may be synthetic. In designing synthetic transmembrane domains, amino acids favoring alpha-helical structures are preferred. Transmembrane domains useful in construction of CARs are comprised of approximately 10, 11, 12, 13, 14, 15, 16, 17, 18, 19, 20, 21, 22, 22, 23, or 24 amino acids favoring the formation having an alpha-helical secondary structure. Amino acids having a to favor alpha-helical conformations are well known in the art. See, e.g Pace, et al. (1998) Biophysical Journal 75: 422-427. Amino acids that are particularly favored in alpha helical conformations include methionine, alanine, leucine, glutamate, and lysine. In some embodiments, the CAR transmembrane domain may be derived from the transmembrane domain from type I membrane spanning proteins, such as CD3, CD4, CD8, CD28, etc.

The cytoplasmic domain of the CAR polypeptide comprises one or more intracellular signal domains. In one embodiment, the intracellular signal domains comprise the cytoplasmic sequences of the T-cell receptor (TCR) and co-receptors that initiate signal transduction following antigen receptor engagement and functional derivatives and sub-fragments thereof. A cytoplasmic signaling domain, such as those derived from the T cell receptor zeta-chain, is employed as part of the CAR in order to produce stimulatory signals for T lymphocyte proliferation and effector function following engagement of the chimeric receptor with the target antigen. Examples of cytoplasmic signaling domains include but are not limited to the cytoplasmic domain of CD27, the cytoplasmic domain S of CD28, the cytoplasmic domain of CD137 (also referred to as 4-1BB and TNFRSF9), the cytoplasmic domain of CD278 (also referred to as ICOS), p110α, β, or δ catalytic subunit of PI3 kinase, the human CD3 chain, cytoplasmic domain of CD134 (also referred to as OX40 and TNFRSF4), FccR1γ and β chains, MB1 (Igα) chain, B29 (IGβ) chain, etc.), CD3 polypeptides (δ, Δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28.

In some embodiments, the CAR may also provide a co-stimulatory domain. The term “co-stimulatory domain”, refers to a stimulatory domain, typically an endodomain, of a CAR that provides a secondary non-specific activation mechanism through which a primary specific stimulation is propagated. The co-stimulatory domain refers to the portion of the CAR which enhances the proliferation, survival or development of memory cells. Examples of co-stimulation include antigen nonspecific T cell co-stimulation following antigen specific signaling through the T cell receptor and antigen nonspecific B cell co-stimulation following signaling through the B cell receptor. Co-stimulation, e.g., T cell co-stimulation, and the factors involved have been described in Chen & Flies. (2013) Nat Rev Immunol 13(4):227-42. In some embodiments of the present disclosure, the CSD comprises one or more of members of the TNFR superfamily, CD28, CD137 (4-1BB), CD134 (OX40), Dap10, CD27, CD2, CD5, ICAM-1, LFA-1 (CD11a/CD18), Lck, TNFR-I, TNFR-II, Fas, CD30, CD40 or combinations thereof.

CARs are often referred to as first, second, third or fourth generation. The term first-generation CAR refers to a CAR wherein the cytoplasmic domain transmits the signal from antigen binding through only a single signaling domain, for example a signaling domain derived from the high-affinity receptor for IgE FcεR1□ or the CD3ζ chain. The domain contains one or three immunoreceptor tyrosine-based activating recognition motif(s) [ITAM(s)] for antigen-dependent T-cell activation. The ITAM-based activating signal endows T-cells with the ability to lyse the target tumor cells and secret cytokines in response to antigen binding. Second-generation CARs include a co-stimulatory signal in addition to the CD3ζ signal. Coincidental delivery of the delivered co-stimulatory signal enhances cytokine secretion and antitumor activity induced by CAR-transduced T-cells. The co-stimulatory domain is usually be membrane proximal relative to the CD3 domain. Third-generation CARs include a tripartite signaling domain, comprising for example a CD28, CD3ζ, OX40 or 4-1BB signaling region. In fourth generation, or “armored car” CAR T-cells are further modified to express or block molecules and/or receptors to enhance immune activity such as the expression of IL-12, IL-18, IL-7, and/or IL-10; 4-1BB ligand, CD-40 ligand.

Examples of intracellular signaling domains comprising may be incorporated into the CAR of the present invention include (amino to carboxy): CD3ζ; CD28-41BB-CD3ζ; CD28-OX40- CD3ζ; CD28-41BB-CD3ζ; 41BB-CD-28-CD3ζ and 41BB-CD3ζ.

The term CAR used herein also includes CAR variants including but not limited split CARs, ON-switch CARS, bispecific or tandem CARs, inhibitory CARs (iCARs) and induced pluripotent stem (iPS) CAR-T cells.

The term “Split CARs” refers to CARs wherein the extracellular portion, the ABD and the cytoplasmic signaling domain of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR molecules and derivatives thereof (i.e., CAR variants) are described, e.g., in PCT Application Nos. US2014/016527, US1996/017060, US2013/063083; Fedorov et al. Sci Transl Med (2013);5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

The term “bispecific or tandem CARs” refers to CARs which include a secondary CAR binding domain that can either amplify or inhibit the activity of a primary CAR.

The term “inhibitory chimeric antigen receptors” or “iCARs” are used interchangeably herein to refer to a CAR where binding iCARs use the dual antigen targeting to shut down the activation of an active CAR through the engagement of a second suppressive receptor equipped with inhibitory signaling domains of a secondary CAR binding domain results in inhibition of primary CAR activation. Inhibitory CARs (iCARs) are designed to regulate CAR-T cells activity through inhibitory receptors signaling modules activation. This approach combines the activity of two CARs, one of which generates dominant negative signals limiting the responses of CAR-T cells activated by the activating receptor. iCARs can switch off the response of the counteracting activator CAR when bound to a specific antigen expressed only by normal tissues. In this way, iCARs-T cells can distinguish cancer cells from healthy ones, and reversibly block functionalities of transduced T cells in an antigen-selective fashion. CTLA-4 or PD-1 intracellular domains in iCARs trigger inhibitory signals on T lymphocytes, leading to less cytokine production, less efficient target cell lysis, and altered lymphocyte motility.

The term “tandem CAR” or “TanCAR” refers to CARs which mediate bispecific activation of T cells through the engagement of two chimeric receptors designed to deliver stimulatory or costimulatory signals in response to an independent engagement of two different tumor associated antigens.

Typically, the chimeric antigen receptor T-cells (CAR-T cells) are T-cells which have been recombinantly modified by transduction with an expression vector encoding a CAR in substantial accordance with the teaching above.

### Kits

Also provided in this disclosure are kits that can be used to activate an immune cell. In some embodiments a vector comprising a coding sequence that encodes the modified human CD122 is provided, where the coding sequence is operably linked to a promoter active in the desired cell. Various vectors are known in the art and can be used for this purpose, e.g., viral vectors, plasmid vectors, minicircle vectors, which can be integrated into the target cell genome, or can be episomally maintained. The modified CD122-encoding vector may be provided in a kit, combined with a vector encoding a cytokine that binds to and activates the receptor. In some embodiments the coding sequence for the cytokine is operably linked to a high expression promoter, and may be optimized for production. In other embodiments, a kit is provided in which the vector encoding the orthogonal receptor is provided with a purified composition of the cognate cytokine, e.g. in a unit dose, packaged for administration to a patient.

### Ex Vivo Activation Methods

In some embodiments, this disclosure provides a method of stimulating an engineered immune cell expressing a modified human CD122 comprising one or more STAT3 binding motifs, the method comprising contacting the immune cell with a human IL2 polypeptide, which bind to the modified human CD122, thereby stimulating the engineered immune cells.

Also provided are methods to selectively activate engineered immune cells expressing a modified human orthogonal CD122 from a mixture of cell population comprising the engineered immune cells and native immune cells, the method comprises contacting the mixture of immune cells with a orthogonal human IL2, which specifically bind to the modified orthogonal human CD122, thereby selectively activating the engineered immune cells.

### Therapeutic Methods

In some cases, a therapeutic method is provided, the method comprising introducing to a subject in need thereof a population of engineered cells expressing a modified human CD122 (e.g., a modified orthogonal human CD122) as provided in this disclosure. The population of engineered cells can be engineered ex vivo and is usually autologous or allogeneic with respect to the subject. Following the introduction of the population of engineered cells, the introduced engineered cell population is contacted with cognate IL2 ligand (e.g., an IL2 ortholog) in vivo.

In some cases, the subject has cancer and the engineered immune cells are CD8+ T cells expressing the modified human CD122 (e.g., modified orthogonal human CD122). In some cases, the subject has autoimmune disease and the immune cells are Treg cells expressing the modified human CD122. In some cases, the engineered immune cells are CAR-T cells (e.g., CD8+ T cells or Treg cells expressing one or more CARs).

### Introducing Immune Cells Expressing Modified Human CD122

In some embodiments, an engineered T cell is allogeneic with respect to the individual that is treated. Graham et al. (2018) Cell 7(10) E155. In some embodiments an allogeneic engineered T cell is fully HLA matched. However not all patients have a fully matched donor and a cellular product suitable for all patients independent of HLA type provides an alternative.

Because the cell product may consist of a subject's own T-cells, the population of the cells to be administered to the subject is necessarily variable, and the response to such agents can vary. Consequently identifying the optimal concentration of the cells involves the ongoing monitoring and management of therapy related toxicities. Usually, at least 1□106 cells/kg will be administered, at least 1□107 cells/kg, at least 1□108 cells/kg, at least 1□109 cells/kg, at least 1□1010 cells/kg, or more, usually being limited by the number of T cells that are obtained during collection. In some cases patients receive a course of pharmacologic immunosuppression or B cell depletion prior to the administration of the CAR-T cell treatment. The engineered cells may be infused to the subject in any physiologically acceptable medium by any convenient route of administration, normally intravascularly, although they may also be introduced by other routes, where the cells may find an appropriate site for growth.

If the T cells are allogeneic T cells, such cells may be modified to reduce graft versus host disease. For example, the engineered cells of the present invention may be TCRαβ receptor knock-outs achieved by gene editing techniques. TCRαβ is a heterodimer and both alpha and beta chains need to be present for it to be expressed. A single gene codes for the alpha chain (TRAC), whereas there are 2 genes coding for the beta chain, therefore TRAC loci KO has been deleted for this purpose. A number of different approaches have been used to accomplish this deletion, e.g. CRISPR/Cas9; meganuclease; engineered I-CreI homing endonuclease, etc. See, for example, Eyquem et al. (2017) Nature 543:113-117, in which the TRAC coding sequence is replaced by a CAR coding sequence; and Georgiadis et al. (2018) Mol. Ther. 26:1215-1227, which linked CAR expression with TRAC disruption by clustered regularly interspaced short palindromic repeats (CRISPR)/Cas9 without directly incorporating the CAR into the TRAC loci. An alternative strategy to prevent GVHD modifies T cells to express an inhibitor of TCRαβ signaling, for example using a truncated form of CD3 as a TCR inhibitory molecule.

### Administering Orthogonal IL2

Following introduction of the population of engineered immune cells expressing a modified human CD122, an cognate IL2 ligand may be provided to a subject by the administration of a nucleic acid construct encoding the cognate IL2 ligand to the subject to achieve continuous exposure of the subject to the cognate IL2 ligand. In some cases, the engineered immune cells express a modified orthogonal human CD122, and a recombinant vector encoding a cognate orthogonal IL2 is introduced to provide for extended delivery of the orthogonal IL2 to the subject and prolonged activation of the corresponding cells engineered to express the cognate orthogonal receptor associated with such orthogonal IL2. Non-Viral:

In one embodiment, the orthogonal IL2 may be administered to a subject in the form of nucleic acid expression construct for the orthogonal IL2 in a non-viral vector may be provided in a non-viral delivery system. Non-viral delivery systems are typically complexes to facilitate transduction of the target cell with a nucleic acid cargo wherein the nucleic acid is complexed with agents such as cationic lipids (DOTAP, DOTMA), surfactants, biologicals (gelatin, chitosan), metals (gold, magnetic iron) and synthetic polymers (PLG, PEI, PAMAM). Numerous embodiments of non-viral delivery systems are well known in the art including lipidic vector systems (Lee et al. (1997) Critical Reviews of Therapeutic Drug Carrier Systems 14:173-206); polymer coated liposomes (Marin et al., U.S. Pat. No. 5,213,804, issued May 25, 1993; Woodle, et al., U.S. Pat. No. 5,013,556, issued May 7, 1991); cationic liposomes (Epand et al., U.S. Pat. No. 5,283,185, issued Feb. 1, 1994; Jessee, J. A., U.S. Pat. No. 5,578,475, issued Nov. 26, 1996; Rose et al, U.S. Pat. No. 5,279,833, issued Jan. 18, 1994; Gebeyehu et al., U.S. Pat. No. 5,334,761, issued Aug. 2, 1994). In one embodiment, the nucleic acid sequence in the non-viral vector system encoding the IL2 receptor is under control of a regulatable promoter, inducible promoter, tissue specific or tumor specific promoter, or temporally regulated promoter.

### Viral Vectors:

In another embodiment, orthogonal IL2 may be administered to a subject in the form of nucleic acid expression construct in viral vector encoding the orthogonal IL2. The terms “viral vector” and “virus” are used interchangeably herein to refer to any of the obligate intracellular parasites having no protein-synthesizing or energy-generating mechanism. The viral genome may be RNA or DNA contained with a coated structure of protein of a lipid membrane. The terms virus(es) and viral vector(s) are used interchangeably herein. The viruses useful in the practice of the present invention include recombinantly modified enveloped or nonenveloped DNA and RNA viruses, preferably selected from baculoviridiae, parvoviridiae, picornoviridiae, herpesviridiae, poxviridae, or adenoviridiae. The viruses are modified by recombinant DNA techniques to include expression of exogenous transgenes (e.g. a nucleic acid sequence encoding the orthogonal IL2) and may be engineered to be replication deficient, conditionally replicating or replication competent. Minimal vector systems in which the viral backbone contains only the sequences need for packaging of the viral vector and may optionally include a transgene expression cassette may also be employed. The term “replication deficient” refers to vectors that are highly attenuated for replication in a wild type mammalian cell. In order to produce such vectors in quantity, a producer cell line is generally created by co-transfection with a helper virus or genomically modified to complement the missing functions. The term “replication competent viral vectors” refers to a viral vector that is capable of infection, DNA replication, packaging and lysis of an infected cell. The term “conditionally replicating viral vectors” is used herein to refer to replication competent vectors that are designed to achieve selective expression in particular cell types. Such conditional replication may be achieved by operably linking tissue specific, tumor specific or cell type specific or other selectively induced regulatory control sequences to early genes (e.g., the E1 gene of adenoviral vectors). Infection of the subject with the recombinant virus or non-viral vector can provide for long term expression of the orthogonal IL2 in the subject and provide continuous selective maintenance of the engineered T cells expressing the CD122 orthogonal receptor. In one embodiment, the nucleic acid sequence in the viral vector system encoding the IL2 receptor is under control of a regulatable promoter, inducible promoter, tissue specific or tumor specific promoter, or temporally regulated promoter.

### Pharmaceutical Formulations

Engineered T cells can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The cells can be administered by any suitable means, usually parenteral. Parenteral infusions include intramuscular, intravenous (bolus or slow infusion), intraarterial, intraperitoneal, intrathecal or subcutaneous administration. In the typical practice, the engineered T cells are infused to the subject in a physiologically acceptable medium, normally intravascularly, although they may also be introduced into any other convenient site, where the cells may find an appropriate site for growth. Usually, at least 1×105 cells/kg will be administered, at least 1×106 cells/kg, at least 1×107 cells/kg, at least 1×108 cells/kg, at least 1×109 cells/kg, or more, usually being limited by the number of T cells that are obtained during collection.

For example, typical ranges for the administration of the engineered immune cells for use in the practice of the present methods range from about 1×105 to 5×108 viable cells per kg of subject body weight per course of therapy. Consequently, adjusted for body weight, typical ranges for the administration of viable cells in human subjects ranges from approximately 1×106 to approximately 1×1013 viable cells, alternatively from approximately 5×106 to approximately 5×1012 viable cells, alternatively from approximately 1×107 to approximately 1×1012 viable cells, alternatively from approximately 5×107 to approximately 1×1012 viable cells, alternatively from approximately 1×108 to approximately 1×1012 viable cells, alternatively from approximately 5×108 to approximately 1×1012 viable cells, alternatively from approximately 1×109 to approximately 1×1012 viable cells per course of therapy. In one embodiment, the dose of the cells is in the range of 2.5-5×109 viable cells per course of therapy.

A course of therapy may be a single dose or in multiple doses over a period of time. In some embodiments, the cells are administered in a single dose. In some embodiments, the cells are administered in two or more split doses administered over a period of 1, 2, 3, 4, 5, 6, 7, 8, 9, 10, 11, 12, 13, 14, 21, 28, 30, 60, 90, 120 or 180 days. The quantity of engineered cells administered in such split dosing protocols may be the same in each administration or may be provided at different levels. Multi-day dosing protocols over time periods may be provided by the skilled artisan (e.g. physician) monitoring the administration of the cells taking into account the response of the subject to the treatment including adverse effects of the treatment and their modulation as discussed above.

The compositions and methods of the present disclosure also provide a method for the treatment of a subject with a T cell therapy (especially CART cell therapy) in the absence of prior lymphodepletion. Lymphodepletion is typically performed in a subject in conjunction with CART cell therapy because the subsequent administration of the mixed cell population and the administration of non-specific agents (e.g., IL2) to expand the engineered cell population in the subject in combination with the administration of the cell therapy product acts results in significant systemic toxicity (including cytokine release syndrome or “cytokine storm”) arising from the widespread proliferation and activation of immune cells by administration of agents that result in widespread activation as well as the presence of a substantial fraction of non-engineered cells in the cell therapy product itself. The methods and compositions of the present disclosure obviate this significant hurdle by both (or either) providing a substantially purified population of engineered cells largely devoid of contamination by non-engineered cells when the foregoing ex vivo method is employed and/or the selective activation and expansion of the engineered T cells with the orthogonal IL2 of the present invention which provide substantially reduced off-target effects of non-specific proliferative agents such as IL2.

For example, in the current clinical practice of CAR-T cell therapy, CAR-T cells are commonly administered in combination with lymphodepletion (e.g., by administration of Alemtuzumab (monoclonal anti-CD52), purine analogs, and the like) to facilitate expansion of the CAR-T cells to prior to host immune recovery. In some embodiments, the CAR-T cells may be modified for resistance to Alemtuzumab. In one aspect of the invention, the lymphodepletion currently employed in association with CAR-T therapy may be obviated or reduced by the orthogonal ligand expressing CAR-Ts of the present invention. As noted above, the lymphodepletion is commonly employed to enable expansion of the CAR-T cells. However, the lymphodepletion is also associated with major side effects of CAR-T cell therapy. Because the orthogonal ligand provides a means to selectively expand a particular T-cell population, the need for lymphodepletion prior to administration of the orthogonal ligand expressing CAR-Ts may be reduced. The present methods enable the practice of CAR-T cell therapy without or with reduced lymphodepletion prior to administration of the orthogonal ligand expressing CAR-Ts.

In one embodiment, the present disclosure provides a method of treating a subject suffering from a disease, disorder or condition amendable to treatment with CAR-T cell therapy (e.g. cancer) by the administration of a orthogonal ligand expressing CAR-Ts in the absence of lymphodepletion prior to administration of the orthogonal ligand CAR-Ts. In one embodiment, the present disclosure provides for a method of treatment of a mammalian subject suffering from a disease, disorder associated with the presence of an aberrant population of cells (e.g. a tumor) said population of cells characterized by the expression of one or more surface antigens (e.g. tumor antigen(s)), the method comprising the steps of (a) obtaining a biological sample comprising T-cells from the individual; (b) enriching the biological sample for the presence of T-cells; (c) transfecting the T-cells with one or more expression vectors comprising a nucleic acid sequence encoding a CAR and a nucleic acid sequence encoding an orthogonal CD122 receptor, the antigen targeting domain of the CAR being capable of binding to at least one antigen present on the aberrant population of cells; (d) expanding the population of the orthogonal receptor expressing CAR-T cells ex vivo with an orthogonal IL2; (e) administering a pharmaceutically effective amount of the orthogonal receptor expressing CAR-T cells to the mammal; and (f) modulating the growth of the orthogonal CD122 receptor expressing CAR-T cells by the administration of a therapeutically effective amount of an orthogonal IL2 that binds selectively to the orthogonal CD122 receptor expressed on the CAR-T cell. In one embodiment, the foregoing method is associated with lymphodepletion or immunosuppression of the mammal prior to the initiation of the course of CAR-T cell therapy. In another embodiment, the foregoing method is practiced in the absence of lymphodepletion and/or immunosuppression of the mammal.

### Therapeutic Combinations:

The compositions and methods of the present disclosure may be combined with additional therapeutic agents. For example, when the disease, disorder or condition to be treated is a neoplastic disease (e.g. cancer) the methods of the present disclosure may be combined with conventional chemotherapeutic agents or other biological anti-cancer drugs such as checkpoint inhibitors (e.g. PD1 or PDL1 inhibitors) or therapeutic monoclonal antibodies (e.g. Avastin, Herceptin).

Examples of chemical agents identified in the art as useful in the treatment of neoplastic disease, include without limitation, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

The compositions of the present disclosure may be administered in combination with one or more additional therapeutic agents selected from the group consisting of tyrosine-kinase inhibitors, such as Imatinib mesylate (marketed as Gleevec®, also known as STI-571), Gefitinib (Iressa®, also known as ZD1839), Erlotinib (marketed as Tarceva®), Sorafenib (Nexavar®), Sunitinib (Sutent®), Dasatinib (Sprycel®), Lapatinib (Tykerb®), Nilotinib (Tasigna®), and Bortezomib (Velcade®), Jakafi® (ruxolitinib); Janus kinase inhibitors, such as tofacitinib; ALK inhibitors, such as crizotinib; Bcl-2 inhibitors, such as obatoclax, venclexta, and gossypol; FLT3 inhibitors, such as midostaurin (Rydapt®), IDH inhibitors, such as AG-221, PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel®), everolimus (Afinitor®), Vemurafenib (Zelboraf®), Trametinib (Mekinist), and Dabrafenib (Tafinlar®).

In some embodiments, particularly where the tumor antigen binding portion of the CAR is directed against BCMA, the engineered CAR-T cell is administered in combination with a γ-Secretase Inhibitor (GSI) as described in Pont, et al. (2019) “γ-secretase inhibition increases efficacy of BCMA-specific chimeric antigen receptor T cells in multiple myeloma” Blood doi.org/10.1182/blood.2019000050.

Examples of biological agents identified in the art as useful in the treatment of neoplastic disease, include without limitation, cytokines or cytokine antagonists such as IL-12, INFα, or anti-epidermal growth factor receptor, radiotherapy, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foregoing as practiced in known chemotherapeutic treatment regimens readily appreciated by the skilled clinician in the art.

Tumor specific monoclonal antibodies that can be administered in combination with an engineered cell may include, without limitation, Rituximab (marketed as MabThera or Rituxan), Alemtuzumab, Panitumumab, Ipilimumab (Yervoy), etc.

In some embodiments the compositions and methods of the present disclosure may be combined with immune checkpoint therapy. Examples of immune checkpoint therapies include inhibitors of the binding of PD1 to PDL1 and/or PDL2. PD1 to PDL1 and/or PDL2 inhibitors are well known in the art. Examples of commercially available monoclonal antibodies that interfere with the binding of PD1 to PDL1 and/or PDL2 include nivolumab (Opdivo®, BMS-936558, MDX1106, commercially available from BristolMyers Squibb, Princeton N.J.), pembrolizumab (Keytruda® MK-3475, lambrolizumab, commercially available from Merck and Company, Kenilworth N.J.), and atezolizumab (Tecentriq®, Genentech/Roche, South San Francisco Calif.). Additional examples of PD1 inhibitory antibodies include but are not limited to durvalumab (MEDI4736, Medimmune/AstraZeneca), pidilizumab (CT-011, CureTech), PDR001 (Novartis), BMS-936559 (MDX1105, Bristol Myers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011. Additionally, small molecule PD1 to PDL1 and/or PDL2 inhibitors are known in the art. See, e.g. Sasikumar, et al as WO2016142833A1 and Sasikumar, et al. WO2016142886A2, BMS-1166 and BMS-1001 (Skalniak, et al (2017) Oncotarget 8(42): 72167-72181).

### Example 1. Preparation of CD19 CAR_T2A_hoRb and CD19 CAR_T2A_hoRB_GGYRHQ T Cells

The CD19 chimeric antigen receptor protein used in this study (hereinafter referred to as “CD19_28z”) had the following structure: a GMCSF receptor signal peptide, the FMC63 anti-CD19 scFv, a AAA linker, a CD28 hinge/transmembrane/costimulatory domain and a CD3zeta. The amino acid sequence of the CD19_28z is as follows:

(SEQ ID NO: 24)
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITGSTSGSGKPGSGEGSTKGEVK
LQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGG
SYAMDYWGQGTSVTVSSAAAIEVMYPPPYLDNEKSNGTIIHVKGKHLCPS
PLFPGPSKPFWVLVVVGGVLACYSLLVTVAFIIFWVRSKRSRLLHSDYMN
MTPRRPGPTRKHYQPYAPPRDFAAYRSRVKFSRSADAPAYQQGQNQLYNE
LNLGRREEYDVLDKRRGRDPEMGGKPRRKNPQEGLYNELQKDKMAEAYSE
IGMKGERRRGKGHDGLYQGLSTATKDTYDALHMQALPPR

The orthogonal T cells used in the conduct of these were generated by techniques known in the art by transfecting isolated T cells with a lentiviral vector the foregoing CD19_28z CAR, T2A linker polypeptide and human orthogonal CD122 orthogonal receptor (hoCD122) having the amino acid sequence:

(SEQ ID NO: 25)
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITGSTSGSGKPGSGEGSTKGEVK
LQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGG
SYAMDYWGQGTSVTVSSAAAIEVMYPPPYLDNEKSNGTIIHVKGKHLCPS
PLFPGPSKPFWVLVVVGGVLACYSLLVTVAFIIFWVRSKRSRLLHSDYMN
MTPRRPGPTRKHYQPYAPPRDFAAYRSRVKFSRSADAPAYQQGQNQLYNE
LNLGRREEYDVLDKRRGRDPEMGGKPRRKNPQEGLYNELQKDKMAEAYSE
IGMKGERRRGKGHDGLYQGLSTATKDTYDALHMQALPPGSGEGRGSLLTC
GDVEENPGPMAAPALSWRLPLLILLLPLATSWASAAVNGTSQFTCFYNSR
ANISCVWSQDGALQDTSCQVHAWPDRRRWNQTCELLPVSQASWACNLILG
APDSQKLTTVDIVTLRVLCREGVRWRVMAIQDFKPFENLRLMAPISLQVV
HVETHRCNISWEISQASDFFERHLEFEARTLSPGHTWEEAPLLTLKQKQE
WICLETLTPDTQYEFQVRVKPLQGEFTTWSPWSQPLAFRTKPAALGKDTI
PWLGHLLVGLSGAFGFIILVYLLINCRNTGPWLKKVLKCNTPDPSKFFSQ
LSSEHGGDVQKWLSSPFPSSSFSPGGLAPEISPLEVLERDKVTQLLLQQD
KVPEPASLSSNHSLTSCFTNQGYFFFHLPDALEIEACQVYFTYDPYSEED
PDEGVAGAPTGSSPQPLQPLSGEDDAYCTFPSRDDLLLFSPSLLGGPSPP
STAPGGSGAGEERMPPSLQERVPRDWDPQPLGPPTPGVPDLVDFQPPPEL
VLREAGEEVPDAGPREGVSFPWSRPPGQGEFRALNARLPLNTDAYLSLQE
LQGQDPTHLV

also referred to as CD19 CAR_T2A_hoRb For hoRB with the wild type ICD, and for hoRB comprising the amino acid sequence:

(SEQ ID NO: 26)
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITGSTSGSGKPGSGEGSTKGEVK
LQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGG
SYAMDYWGQGTSVTVSSAAAIEVMYPPPYLDNEKSNGTIIHVKGKHLCPS
PLFPGPSKPFWVLVVVGGVLACYSLLVTVAFIIFWVRSKRSRLLHSDYMN
MTPRRPGPTRKHYQPYAPPRDFAAYRSRVKFSRSADAPAYQQGQNQLYNE
LNLGRREEYDVLDKRRGRDPEMGGKPRRKNPQEGLYNELQKDKMAEAYSE
IGMKGERRRGKGHDGLYQGLSTATKDTYDALHMQALPPGSGEGRGSLLTC
GDVEENPGPMAAPALSWRLPLLILLLPLATSWASAAVNGTSQFTCFYNSR
ANISCVWSQDGALQDTSCQVHAWPDRRRWNQTCELLPVSQASWACNLILG
APDSQKLTTVDIVTLRVLCREGVRWRVMAIQDFKPFENLRLMAPISLQVV
HVETHRCNISWEISQASDFFERHLEFEARTLSPGHTWEEAPLLTLKQKQE
WICLETLTPDTQYEFQVRVKPLQGEFTTWSPWSQPLAFRTKPAALGKDTI
PWLGHLLVGLSGAFGFIILVYLLINCRNTGPWLKKVLKCNTPDPSKFFSQ
LSSEHGGDVQKWLSSPFPSSSFSPGGLAPEISPLEVLERDKVTQLLLQQD
KVPEPASLSSNHSLTSCFTNQGYFFFHLPDALEIEACQVYFTYDPYSEED
PDEGVAGAPTGSSPQPLQPLSGEDDAYCTFPSRDDLLLFSPSLLGGPSPP
STAPGGSGAGEERMPPSLQERVPRDWDPQPLGPPTPGVPDLVDFQPPPEL
VLREAGEEVPDAGPREGVSFPWSRPPGQGEFRALNARLPLNTDAYLSLQE
LQGQDPTHLVGGYRHQ

The use of the T2A linker enables the use of a single lentiviral plasmid to generate the orthogonal CAR-T cells.

Primary CD4 and CD8 enriched T cells were stimulated with anti-CD3 and anti-CD28 antibodies and grown in WT IL-2 during until 2 days post-transduction. Cells were transduced with lentivirus encoding CD19 CAR_T2A_hoRb or CD19 CAR_T2A hoRb_GGYRHQ. 2 days later, cells were washed and switched to T cell growth media including an the 40Kd PEGylated orthogonal ligand STK-009 (100 nM). Cells were maintained at a cell density of 1E6 cell/ml of T cell media+STK-009 until Day 10 when cells were harvested.

### Example 2: Orthogonal Ligand STK-009

An orthogonal cognate ligand for the hoCD122 receptor used in these studies (hereinafter STK-009) is a compound of the hIL2 mutein comprising a deletion of the N-terminal alanine residue and comprising the amino acid substitutions L18R, Q22E and Q126K (numbered in accordance with mature wild type hIL2) further modified to by addition to the N-terminal proline residue a 40 kD branched (2×20 kD) PEG molecule with an aldehyde linker a 40 kDa 2-arm branched PEG-aldehyde the 40 kDA PEG-aldehyde comprising two 20 kDA linear PEG molecules (Sunbright® GL2-400AL3, NOF America Corporation, One North Broadway, White Plains, N.Y. 10601 USA.

### Example 3. Phospho-Signaling Assay

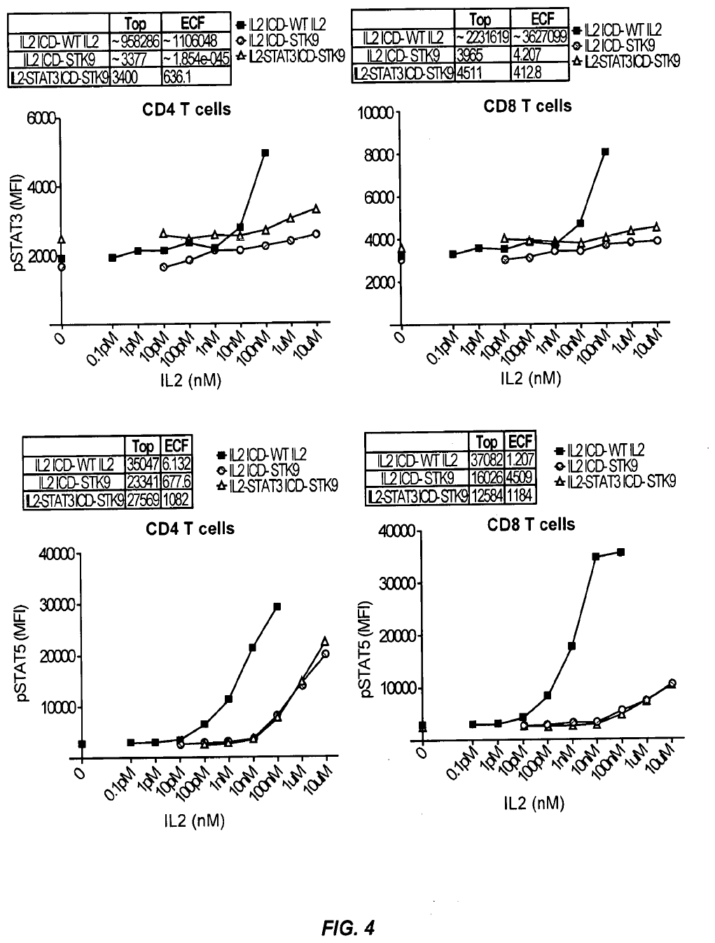

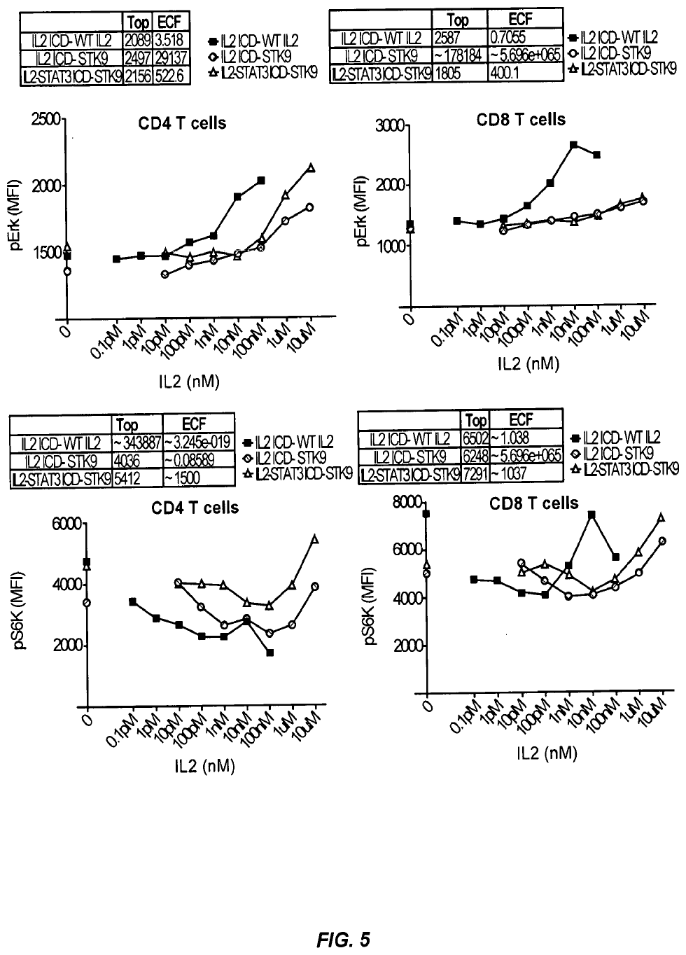

T cells stably transduced with CD19 CAR T2A_hoRb or CD19 CAR T2A hoRb_GGYRHQ were rested in RPMI media+10% FBS for 1 hour. Cells were then stimulated with a dose titration of either WT IL-2 or STK-009 for 20 minutes and stained for various phospho-antigens. Cells were then subjected to flow cytometric analysis. The results of these studies are provided in FIGS. 4 and 5 of the attached drawings. As indicated, the addition of the STAT3 motif to the hoCD122 receptor in CAR T cells results in increased activation of pERK, pS6K, and a higher baseline of pSTAT3.

### Example 4. Cytotoxicity Co-Culture Assay

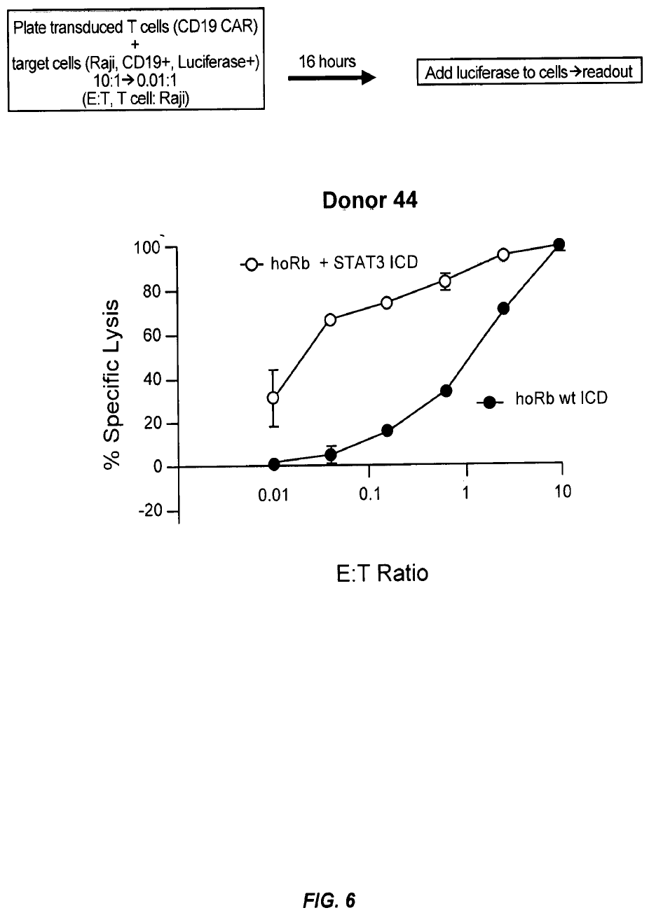

T cells stably transduced with CD19 CAR T2A_hoRb or CD19 CAR T2A hoRb_GGYRHQ were co-cultured with the CD19 target cell line, Raji-luciferase, overnight at the indicated effector to target cell (E:T) ratios. Cell viability was assessed by the conversion of D-luciferin to light and read by a bioluminescent plate reader. Data is shown as specific lysis calculated as 100%× (Spontaneous Death RLU−Test RLU)/(Spontaneous death RLU−Maximal Killing RLU). The results of the study are presented in FIG. 6 of the attached drawings. The data demonstrates that the addition of the STAT3 motif to the ortho receptor in CAR T cells results in superior cytotoxic activity.

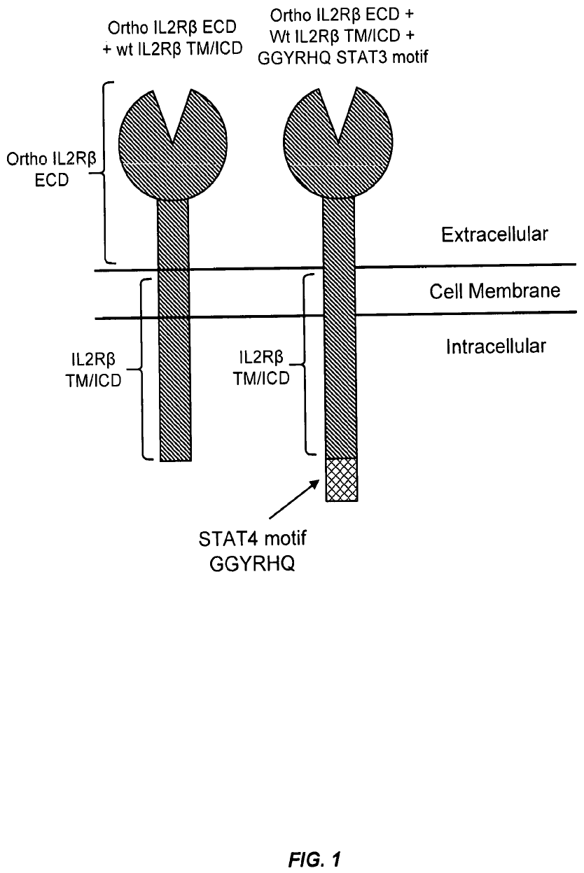

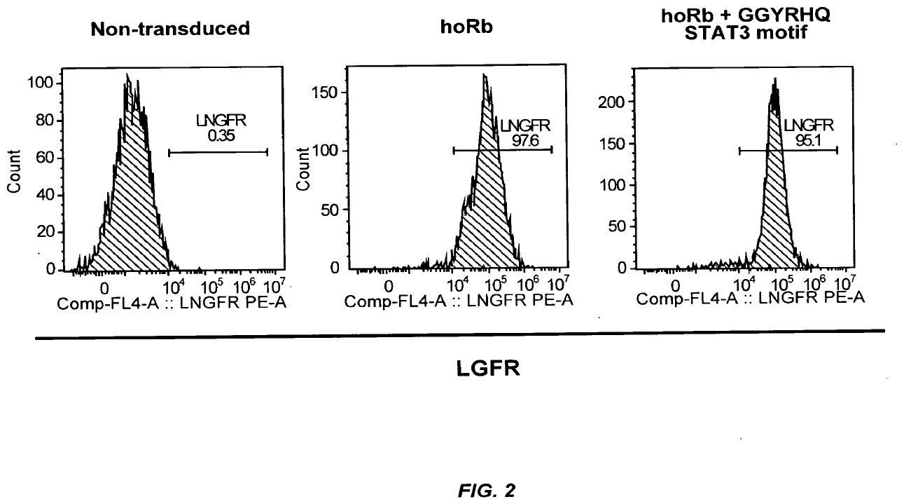

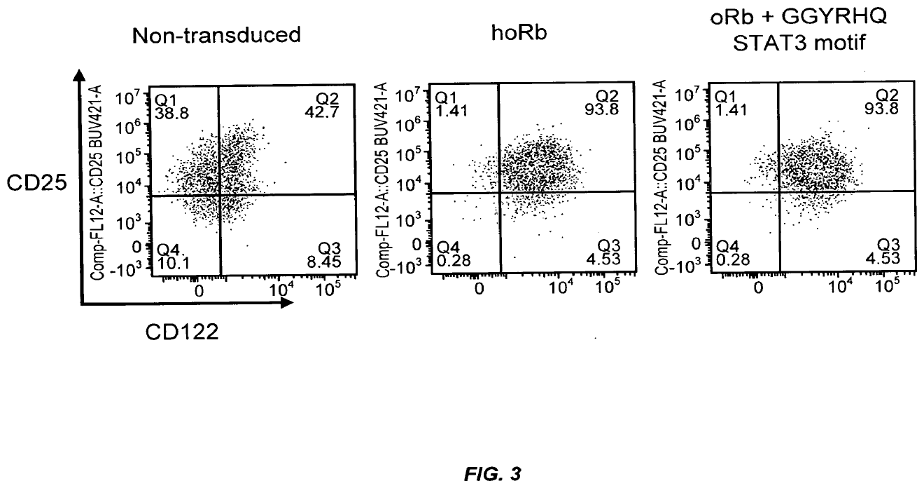

The beneficial functional effects of the addition of the STAT3 signaling domain to intracellular domain of the human ortho Rb (hoRb, hoCD122) receptor were demonstrated in a series of studies to evaluate the effects of cells expressing the wt hoCD122 receptor and a hoCD122 receptor modified to incorporate a STAT3 signaling domain (YRHQ) to the carboxy terminus of the ICD of the hoCD122. A graphical illustration of the structure of the receptors is provided in FIG. 1 of the attached drawings. Cell lines stably expressing the hoRB receptor with the wild type ICD and the STAT3 modified ICD were prepared and tested with wild type IL2 and the orthogonal ligand STK-009 (Example 2). As illustrated in FIGS. 2 and 3, the hoRB receptor with the wild type ICD and the STAT3 modified ICD were functional in the modified cells. The cells expressing the receptors were evaluated for the expression of a variety of phosphor ligands in substantial accordance with the Example 3. The results are provided in FIGS. 4 and 5. As indicated in FIGS. 4 and 5, the addition of the STAT3 motif to the hoCD122 receptor in CAR T cells resulted in increased activation of pERK, pS6K, and a higher baseline levels of pSTAT3.

To evaluate the enhanced efficacy of CAR-T cells expressing the STAT3 modified orthogonal CD122, CAR-T cells were evaluated in a cytotoxicity experiment using Raji tumor cell line. The effect of STAT3 on the cytotoxicity of CD19 CAR T cell constructs comprising orthogonal CD122 (hoRb) receptors with an additional STAT3 signaling motif as compared to CD19 CART cell constructs comprising orthogonal CD122 (hoRb) with the wild type CD122 intracellular domain at various effector:target (E:T; CART:Raji tumor cell) ratios in substantial accordance with the teaching of Example 4. The results of the study are provided in FIG. 6 of the attached drawings. The data provided in FIG. 6 demonstrates the improved cytotoxicity of a CD19 CAR T cell construct expressing an hoRb with an intracellular domain (ICD) modified to contain a STAT3 motif against Raji tumor cells relative to a CD19 CART cell comprising orthogonal CD122 (hoRb) with the wild type CD122 intracellular domain. The foregoing data demonstrates the beneficial properties of the STAT3 motif modified receptors of the present disclosure.

The invention now being fully described, it will be apparent to one of ordinary skill in the art that various changes and modifications can be made without departing from the spirit or scope of the invention.

### Illustrative Sequences:

(native human CD122, mature protein)
SEQ ID NO: 1
AVNGTSQFTC FYNSRANISC VWSQDGALQD TSCQVHAWPD
RRRWNQTCEL LPVSQASWAC NLILGAPDSQ KLTTVDIVTL
RVLCREGVRW RVMAIQDFKP FENLRLMAPI SLQVVHVETH
RCNISWEISQ ASHYFERHLE FEARTLSPGH TWEEAPLLTL
KQKQEWICLE TLTPDTQYEF QVRVKPLQGE FTTWSPWSQP
LAFRTKPAAL GKDTIPWLGH LLVGLSGAFG FIILVYLLIN
CRNTGPWLKK VLKCNTPDPS KFFSQLSSEH GGDVQKWLSS
PFPSSSFSPG GLAPEISPLE VLERDKVTQL LLQQDKVPEP
ASLSSNHSLT SCFTNQGYFF FHLPDALEIE ACQVYFTYDP
YSEEDPDEGV AGAPTGSSPQ PLQPLSGEDD AYCTFPSRDD
LLLFSPSLLG GPSPPSTAPG GSGAGEERMP PSLQERVPRD
WDPQPLGPPT PGVPDLVDFQ PPPELVLREA GEEVPDAGPR
EGVSFPWSRP PGQGEFRALN ARLPLNTDAY LSLQELQGQD PTHL
SEQ ID NO: 2
APTSSSTKKT QLQLEHLLLD LQMILNGINN YKNPKLTRML
TFKFYMPKKA TELKHLQCLE EELKPLEEVL NLAQSKNFHL
RPRDLISNIN VIVLELKGSE TTFMCEYADE TATIVEFLNR
WITFCQSIIS TLT
SEQ ID NO: 3
GGYLRQ
SEQ ID NO: 4
GGYLKQ
SEQ ID NO: 5
GGYRHQ
SEQ ID NO: 6
GGYLRQ
SEQ ID NO: 7
GGYFKQ
SEQ ID NO: 8
GGYLPQ
SEQ ID NO: 9
GGYMPQ
SEQ ID NO: 10
GGYDKPH
SEQ ID NO: 11
YLRQ
SEQ ID NO: 12
YLKQ
SEQ ID NO: 13
YRHQ
SEQ ID NO: 14
YLRQ
SEQ ID NO: 15
YFKQ
SEQ ID NO: 16
YLPQ
SEQ ID NO: 17
YMPQ
SEQ ID NO: 18
YDKPH
SEQ ID NO: 19
YX1X2L
SEQ ID NO: 20
YLSL
SEQ ID NO: 21
YX1X2Q
(native human CD122, full length protein)
The first underlined region is the signal peptide
SEQ ID NO: 22
MAAPALSWRLPLLILLLPLATSWASAAVNGTSQFTCFYNSRANISCVWSQ
DGALQDTSCQVHAWPDRRRWNQTCELLPVSQASWACNLILGAPDSQKLTT
VDIVTLRVLCREGVRWRVMAIQDFKPFENLRLMAPISLQVVHVETHRCNI
SWEISQASHYFERHLEFEARTLSPGHTWEEAPLLTLKQKQEWICLETLTP
DTQYEFQVRVKPLQGEFTTWSPWSQPLAFRTKPAALGKDTIPWLGHLLVG
LSGAFGFIILVYLLINCRNTGPWLKKVLKCNTPDPSKFFSQLSSEHGGDV
QKWLSSPFPSSSFSPGGLAPEISPLEVLERDKVTQLLLQQDKVPEPASLS
SNHSLTSCFTNQGYFFFHLPDALEIEACQVYFTYDPYSEEDPDEGVAGAP
TGSSPQPLQPLSGEDDAYCTFPSRDDLLLFSPSLboLGGPSPPSTAPGGS
GAGEERMPPSLQERVPRDWDPQPLGPPTPGVPDLVDFQPPPELVLREAGE
EVPDAGPREGVSFPWSRPPGQGEFRALNARLPLNTDAYLSLQELQGQDPT
HLV
SEQ ID NO: 23
PTSSSTKKTQLQLSQLLVLLKAILNGINNYKNPKLTRMLTFKFYMPKKAT
ELKHLQCLEEELKPLEEVLNLAQSKNFHLRPRDLISNINVIVLELKGSET
TFMCEYADETATIVEFLNRWITFCQSIISTLT

1. A polynucleotide encoding a modified human CD122, wherein the modified human CD122 comprises one or more STAT3 binding motifs.

2. The polynucleotide of claim 1, wherein the modified human CD122 comprises an orthogonal human CD122 or native human CD122 fused to one or more STAT3 binding motifs.

3. The polynucleotide of claim 2, wherein the orthogonal human CD122 is modified at one or more residues selected from R41, R42, Q70, K71, T73, T74, V75, S132, H133, Y134, F135, E136, and Q214 relative to native human CD122.

4. The polynucleotide of claim 2, wherein the orthogonal human CD122 is modified at H133 and Y134 relative to native human CD122.

5. The polynucleotide of claim 1, wherein the human CD122 is linked to two or three STAT3 binding motifs.

6. The polynucleotide of claim 1, wherein the modified human CD122 comprises a sequence that is at least 90% identical to SEQ ID NO: 1, wherein the modified human CD122 binds to a native IL2 polypeptide or an orthogonal IL2 polypeptide.

7. The polynucleotide of claim 1, wherein the one or more STAT3 binding motifs comprise a sequence of YX₁X2Q, wherein X₁ and X₂ are any amino acid acids.

8. The polynucleotide of claim 7, wherein X₁ is selected from the group consisting of L, R, F, M, and X₂ is selected from the group consisting of R, K, H, and P.

9. The polynucleotide of claim 7, wherein the STAT3 recognition motif is selected from the group consisting of

| (SEQ ID NO: 11) | |
|---|---|
| (a) | YLRQ, |
| (SEQ ID NO: 12) | |
| (b) | YLKQ, |
| (SEQ ID NO: 13) | |
| (c) | YRHQ |
| (SEQ ID NO: 14) | |
| (d) | YLRQ, |
| (SEQ ID NO: 15) | |
| (e) | YFKQ, |
| (SEQ ID NO: 16) | |
| (f) | YLPQ, |
| (SEQ ID NO: 17) | |
| (g) | YMPQ, |
| and | |
| (SEQ ID NO: 18) | |
| (h) | YDKPH. |

10. The polynucleotide of claim 1, wherein the one or more STAT3 binding motifs are fused to a C-terminus of the intracellular domain of the native human CD122 or the orthogonal human CD122, optionally through a linker.

11. The polynucleotide of claim 1, wherein at least one of the STAT3 binding motifs is located between position 355 and position 364 corresponding to native human CD122, and wherein the at least one of the STAT3 recognition motif replaces an amino acid sequence of YFTY, YDPY, or YSEE in native human CD122.

12. The polynucleotide of claim 11, wherein the modified human CD122 is further modified at one or more residues selected from R41, R42, Q70, K71, T73, T74, V75, S132, H133, Y134, F135, E136, and Q214 relative to native human CD122.

13-14. (canceled)

15. An expression vector comprising the polynucleotide of claim 1.

16. A cell comprising the polynucleotide of claim 1 and expressing the modified human CD122.

17. The cell of claim 16, wherein the cell further expresses a chimeric antigen receptor (CAR), and wherein the cell is a human immune cell.

18. The cell of claim 17, wherein the CAR is selected from the group consisting of a CD19 CAR and a BCMA CAR.

19-27. (canceled)

28. A method of stimulating an immune cell expressing a modified human CD122 comprising one or more STAT3 binding motifs, the method comprising contacting the immune cell with a human IL2 polypeptide.

29. The method of claim 28, wherein the stimulating occurs ex vivo.

30. The method of claim 28, wherein the stimulating occurs in vivo.

31-42. (canceled)
